SEQUENCE LISTING (9 sequences)

SEQ ID NO: 1 - DNA, 22 bp, Artificial Sequence
  Description of Artificial Sequence Synthetic primer
gcatgtctggttaggagaaacc

SEQ ID NO: 2 - DNA, 22 bp, Artificial Sequence
  Description of Artificial Sequence Synthetic primer
atgtatgcaatgctgttcttgc

SEQ ID NO: 3 - DNA, 20 bp, Artificial Sequence
  Description of Artificial Sequence Synthetic primer
ccttgaaaaatgccctgaaa

SEQ ID NO: 4 - DNA, 20 bp, Artificial Sequence
  Description of Artificial Sequence Synthetic primer
cttggagagagccacaaagg

SEQ ID NO: 5 - DNA, 16 bp, Artificial Sequence
  Description of Artificial Sequence Synthetic primer
aaagcccagcgactct

SEQ ID NO: 6 - DNA, 21 bp, Artificial Sequence
  Description of Artificial Sequence Synthetic primer
ctaaacggtggtgccatagat

SEQ ID NO: 7 - DNA, 20 bp, Artificial Sequence
  Description of Artificial Sequence Synthetic primer
agtcttcggacgcaagaaaa

SEQ ID NO: 8 - DNA, 20 bp, Artificial Sequence
  Description of Artificial Sequence Synthetic primer
agccaccagagcttttgaga

SEQ ID NO: 9 - protein, 4 aa, Artificial Sequence
  Description of Artificial Sequence Synthetic peptide
GRGD

(19) United States
(12) Patent Application Publication
(10) Pub. No.: US 2014/0349367 A1
(43) Pub. Date: Nov. 27, 2014

(54) Hydrolytically Degradable Micellar Hydrogels

(71) Applicant: University of South Carolina, Columbia, SC (US)

(72) Inventor: Esmaiel Jabbari, Columbia, SC (US)

(73) Assignee: University of South Carolina, Columbia, SC (US)

(21) Appl. No.: 14/260,434

(22) Filed: Apr. 24, 2014

Related U.S. Application Data
(60) Provisional application No. 61/854,438, filed on Apr. 24, 2013.
(60) Provisional application No. 61/854,439, filed on Apr. 24, 2013.

(51) Int. Cl. C12N 5/00; C12N 11/04; C08K 3/20; C12N 5/0775

(52) U.S. Cl. CPC C12N 5/0012; C12N 5/0662; C12N 11/04; C08K 3/20
USPC 435/182; 435/397; 524/832

(57) ABSTRACT

Degradable and biologically inert hydrogel networks are described. The hydrogel networks are crosslinked and based on a biocompatible polymer that is chain extended with hydrophobic segments that include no more than 5 hydrophobic monomers to form a macromonomer that is then crosslinked to form a network that includes individual micelles throughout the crosslinked network. The hydrophobic segments of the macromonomer as well as other potentially toxic materials such as crosslink initiators can be sequestered in the micelles to better control degradation characteristics of the network as well as prevent toxicity to developing cellular structures of the network.

### CROSS REFERENCE TO RELATED APPLICATION

This application claims filing benefit of U.S. Provisional Patent Application Ser. No. 61/854,438 having a filing date of Apr. 24, 2013 titled “Gelation Characteristics and Osteogenic Differentiation of Stromal Cells in Inert Hydrolytically Degradable Micellar Polyethylene Glycol Hydrogels” and U.S. Provisional Patent Application Ser. No. 61/854,439 having a filing date of Apr. 24, 2013 titled “Nanostructure Formation in Polyethylene Glycol Hydrogels Chain Extended Short Hydroxy Acid Segments,” both of which are incorporated herein in their entirety.

### SEQUENCE LISTING

The instant application contains a Sequence Listing which has been submitted electronically in ASCII format and is hereby incorporated by reference in its entirety. Said ASCII copy, created on Jul. 16, 2014, is named USC-408_SL.txt and is 2,321 bytes in size.

### BACKGROUND

Hydrogels are three-dimensional polymeric networks that retain a significant volume of water in their structure without dissolving. Due to their high water content, hydrogels such as those based on polyvinyl alcohol (PVA), polyhydroxyethyl methacrylate (PHEMA), polyethylene glycol (PEG) and polyvinylpyrrolidone (PVP) are used extensively in medicine for soft tissue repair. Due to their high diffusivity of nutrients and biomolecules, hydrogels are also very useful as a matrix materials in tissue engineering, such as for in situ delivery of cells to a regeneration site and controlling the cell fate. Only those hydrogels that degrade and provide free volume for the newly formed tissue are useful in tissue regeneration. Unfortunately, viability and fate of encapsulated cells in hydrogels are generally limited by the toxic effect of gelation and degradation reactions in the matrix. Consequently, natural hydrogels derived from components of the extracellular matrix of biological tissues that physically crosslink and degrade enzymatically are frequently used as the delivery matrix in clinical applications.

Natural hydrogels present difficulties in use however. For instance, minor variation in the sequence distribution of natural gels can dramatically affect the fate of encapsulated cells in the matrix. Natural gels have many cell-interactive ligands and regulatory factors, which make it difficult to tailor these matrices to a particular tissue engineering application. For example, when pure collagen type I matrix has been replaced with type II in a hydrogel, differentiation of multipotent stromal cells (MSCs) changed from an osteogenic to a chondrogenic lineage. Furthermore, due to their low stiffness natural gels are limited by soft tissue compression.

Polyethylene glycol (PEG) hydrogels are inert, non-immunogenic, compatible with stem cells and can be conjugated with bioactive peptides to modify the microenvironment and control cell fate. Due to their inert nature, PEG hydrogels provide enormous flexibility in design and control of the cell microenvironment. The inert nature of PEG can also potentially minimize adsorption and denaturation of proteins as may be synthesized by encapsulated cells, which could otherwise adversely affect the cell fate and function, and can stabilize the active protein by reducing aggregation. Unlike small-molecule monomers that can cross the cell membrane, flexible PEG macromers can crosslink to produce hydrogels with high compressive modulus without adversely affecting the viability of encapsulated cells.

As a result of such beneficial characteristics, PEG hydrogels have been used extensively as a matrix for cell encapsulation to elucidate the effect of physiochemical, mechanical, and biological factors on cell fate in the in vitro microenvironement. Unfortunately, PEG hydrogels are non-degradable, which limits their use as a supporting matrix in regenerative medicine.

One approach to in vivo tissue engineering has included delivering progenitor cells to the regeneration site in an inert matrix, such as a PEG hydrogel, where the encapsulated cells secrete the desired extracellular matrix (ECM). In this approach, the encapsulated progenitor cells, guided by cell-cell interactions and soluble factors, create and reorganize their ECM as they go through lineage commitment, differentiation, and maturation. PEG persistence (non-degradability) at the site of regeneration to provide free volume for tissue formation and remodeling remains an issue, however.

PEG hydrogels can undergo oxidative degradation in the presence of reactive oxygen species secreted by macrophages, activated by the foreign body response. However, degradation by reactive oxygen species is unpredictable and depends on the extent of foreign body response.

Attempts have been made to improve PEG hydrogels. For instance, PEG macromers have been copolymerized with hydroxy acid monomers produce block copolymers that have limited solubility in aqueous solution and self-assemble to form nanoparticles for drug delivery. PEG has also been copolymerized with poly(lactide) (PLA) to impart degradability to PEG macromers. However, due to the hydrophobicity of lactide, these copolymers form thermo-responsive physical gels in aqueous solution with orders of magnitude lower modulus than the covalently crosslinked PEG hydrogels due to entrapment of reactive groups in micellar domains. The degradation and water content of the copolymers can be adjusted by the fraction of hydrophobic lactide segments, but solubility of the copolymers in aqueous solution decreases with increasing lactide content.

What is needed in the art is a method for synthesizing degradable hydrogels as may be used as matrices for cell encapsulation. For instance, there is a need for cell-compatible hydrogels with well-defined tunable physical, mechanical, and biological properties for a wide range of applications in regenerative medicine such as chondrocyte implantation in cartilage regeneration or as cardiac patches to treat heart infarction. Specifically, design and synthesis of hydrogels with reduced toxicity and having hydrolytically degradable links would substantially increase their use as a cell delivery matrix in tissue regeneration.

### SUMMARY

According to one embodiment, disclosed herein is a biocompatible hydrogel network. The network includes a crosslinked macromonomer that is formed of a biocompatible polymer and a hydrophobic segment at the termini of the polymer. Specifically, the hydrophobic segment includes no more than 5 hydrophobic monomers. The hydrogel network includes a micelle, and the micelle includes the crosslinked macromonomer in an orientation such that the hydrophobic segment is sequestered in the core of the micelle.

Also disclosed are methods for forming a crosslinked hydrogel network. For instance, a method can include extending a chain of a biocompatible polymer with a hydrophobic segment to form a macromonomer. The hydrophobic segment includes no more than 5 hydrophobic monomers. The method can also include crosslinking the macromonomer to form the hydrogel network. The crosslinked macromonomer forming a micelle in the hydrogel network. The micelles including a core and the hydrophobic segment of the macromonomer can be sequestered in the core.

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIG. 1 graphically illustrates the effect of the number of monomers per chain-extending segment on the compressive modulus (FIG. 1A), the gelation time (FIG. 1B), the swelling ratio (FIG. 1C) and the sol fraction (FIG. 1D) for several different macromonomers as described herein.

FIG. 2 graphically illustrates the effect of the concentration of crosslinking functionality on the compressive modulus (FIG. 2A), the gelation time (FIG. 2B), the swelling ratio (FIG. 2C), and the sol fraction (FIG. 2D) for several different macromonomers as described herein.

FIG. 3 is a bead representation of aqueous polyethylene glycol/hydroxy acid/acrylate (SPEXA (X=lactide (L), glycolide (G), ε-caprolactone (C), or p-dioxanone (D)) macromonomer, Beads marked SPEGc, EO, G, D, L, C and Ac represent star PEG core, ethylene oxide repeat unit, glycolide, p-dioxanone, lactide, ε-caprolactone repeat unit, and acrylate functional group, respectively.

FIG. 4 illustrates the evolution of the core of the micells in 20% aqueous solutions of SPEXA. Only X and Ac beads are shown for clarity. “n” (column top) is the number of hydroxy acid repeat units. G, D, L and C beads are shown in the rows, as marked.

FIG. 5 illustrates the cross section of the micelles formed in 20% aqueous solutions of SPEXA. “n” is the number of hydroxy acid repeat units. Water beads are not shown for clarity.

FIG. 6 illustrates the effect of the number of degradable hydroxy acid repeat units on each arm on core radius (FIG. 6A), aggregation number (FIG. 6B), number density of micelles (FIG. 6C) and free arm fraction of the micelles (FIG. 6D) in 20% aqueous solutions of SPEXA. Error bars correspond to means±SD for 5 simulation runs.

FIG. 7 illustrates the distribution of initiator molecules for aqueous solution of SPELA-m3 in the simulation box (FIG. 7A) and in the corresponding cross-section of one of the micelles (FIG. 7B). L, Ac, and initiator beads in FIG. 7A and FIG. 7B are shown in different shades. EO and water beads are not shown for clarity. Effect of the number of degradable monomers per arm on simulated Ac-Ac running integration number is provided in FIG. 7C and experimental gelation time of 20% aqueous solutions of SPEXA is shown in FIG. 7D. Error bars in FIG. 7C correspond to means±SD for 5 simulation runs. Error bars in FIG. 7D correspond to mean±SD for 3 experiments.

FIG. 8 illustrates the effect of the number of degradable monomers per arm on ester-W running integration number (FIG. 8A) and predicted hydrolysis rate (FIG. 8B) for 20% aqueous solutions of SPEXA. FIG. 8C presents the effect of the number of lactide (L) monomers per macromonomer on experimental mass loss of SPELA hydrogels. FIG. 8D illustrates the effect of degradable hydroxy acid monomer type on experimental mass loss of SPEXA hydrogels with incubation time. FIG. 8E illustrates the effect of degradable hydroxy acid monomer type on distribution of water beads around SPEXA core of the micelles. FIG. 8F illustrates the effect of number of degradable monomers per arm on experimentally-measured equilibrium water content of SPEXA hydrogels. In FIG. 8A and FIG. 8B, error bars correspond to means±SD for 5 simulation runs. In FIG. 8D, FIG. 8E, and FIG. 8F, error bars correspond to mean±SD for 3 experiments. In FIG. 8E, G, D, L, C, Ac, and water beads are shown in different shades and EO beads are not shown for clarity.

FIG. 9 presents the total collagen (FIG. 9A), ALP activity (FIG. 9B) and calcium content (FIG. 90) of MSCs encapsulated in SPEXA hydrogels with incubation time in osteogenic medium. Also shown is mRNA expression of Col-1 (FIG. 9D), ALP (FIG. 9E) and OC (FIG. 9F) of MSCs encapsulated in SPEXA hydrogels with incubation time in osteogenic medium. * indicates statistically significant difference (p<0.05) between the test group and all other groups at the same time point. Error bars correspond to mean±SD for 3 experiments.

FIG. 10 is a coarse-grained representation of LPELA and SPELA macromonomers, respectively. In a given arm, the length of lactide segment was significantly less than that of EO, leading to micellization and structure formation at the nanoscale.

FIG. 11 presents the 1H-NMR spectrum of SPELA-nL14.8 macromonomer. The inset shows the chemical shifts with peak positions between 5.8 and 6.5 at higher intensity. EO, L, and Ac represent ethylene oxide and lactide repeat units, and acrylate terminal group, respectively.

FIG. 12 illustrates the effect of UV initiator concentration on storage modulus (FIG. 12A) and gelation time (FIG. 12B) of LPELA-nL7.4-M20 and SPELA-nL14.8-M20 hydrogels, Error bars correspond to means±1 SD for n=3. The modulus of SPELA and LPELA gels peaked at 0.6 wt % initiator concentration. Gelation times decreased significantly in the 0.1-0.6 wt % initiator concentration range.

FIG. 13 illustrates the effect of macrmonomer concentration on storage modulus (FIG. 13A) and gelation time (FIG. 13B) of LPELA-nL7.4 and SPELA-nL14.8 hydrogels. Error bars correspond to means±1 SD for n=3. For all concentrations, gelation time of SPELA was lower than LPELA and modulus of SPELA was higher than LPELA. The difference in gelation times of SPELA and LPELA decreased with increasing macromonomer concentration.

FIG. 14 presents the Dissipative Particle Dynamic (DPD) simulation of micellar cores for SPELA-nL14.8-M20 (FIG. 14A); L and Ac beads are shown, while other beads are not shown for clarity. FIG. 14B illustrates a simulated cross-section of one of the micelles in FIG. 14A; water beads are not shown for clarity. FIG. 14C illustrates the intra-molecular running integration number (IN) for Ac-Ac beads as a function of radius around an Ac bead for LPELA-nL7.4 and SPELA-nL14.8 macromonomers in aqueous solution. SPELA macromonomers have significantly higher Ac-Ac integration number than LPELA, leading to shorter gelation times and higher modulus.

FIG. 15 Illustrates the effect of macromonomer concentration on swelling ratio (FIG. 15A) and sol fraction (FIG. 15B) of LPELA-nL7.4 and SPELA-nL14.8 hydrogels. Error bars correspond to means±1 SD for n=3. For all concentrations, swelling ratio and sol fraction of SPELA hydrogel was lower than LPELA. The SPELA-nL14.8 hydrogel with 25% macromonomer concentration had the lowest sol fraction (5%).

FIG. 16 illustrates the effect of number of lactide monomers per macromonomer (nL) on storage modulus (FIG. 16A) and gelation time (FIG. 16B) of LPELA-M20 and SPELA-M20 hydrogels. Error bars correspond to means±1 SD for n=

FIG. 17 illustrates the effect of number of lactide monomers per macromonomer (nL) on swelling ratio (FIG. 17A) and sol fraction (FIG. 17B) of LPELA-M20 and SPELA-M20 hydrogels. Error bars correspond to means±1 SD for n=3. For all nL values, the storage modulus of SPELA hydrogel was higher than LPELA. Gelation time of SPELA hydrogel was higher than LPELA for nL<9 but reduced to below LPELA for nL>9

FIG. 18 presents the effect of the number of lactide monomers per macromonomer (nL) on mass loss of SPELA-M20 (FIG. 18A) and LPELA-M20 (FIG. 18B) hydrogels with incubation time. Error bars correspond to means±1 SD for n There was no significant difference between the mass loss curves of SPELA-nL6.4 and SPELA-nL14.8 (p=0.34) but there was a significant difference between the mass loss of all other SPELA pairs (p<0.05). There was a significant difference between the mass loss of all SPELA pairs (p<0.05).

FIG. 19 includes live (light) and dead image of MSCs 1 h after encapsulation in SPELA-nL3.4 (FIG. 19A), SPELA-nL6.4 (FIG. 19B), SPELA-nL11.6 (FIG. 19C), SPELA-nL14.8 (FIG. 19D) hydrogels (without BMP2). The scale bar is 100 μm. The fraction of viable cells for SPELA-nL0, SPELA-nL3.4, SPELA-nL6.4, SPELA-nL11.6, and SPELA-nL14.8 gels was 92±3, 90±4, 92±4, 94±4, and 91±3, respectively. The number of lactides per macromonomer did not have a significant effect on cell viability.

FIG. 20 illustrates the DNA content (FIG. 20A), ALPase activity (FIG. 20B), and calcium content (FIG. 20C) of MSCs encapsulated in SPELA-14.8 hydrogel. Experimental groups include gels without MSCs incubated in osteogenic media (OM+no MSCs, control), gels with MSCs incubated in basal media (BM, control), gels with MSCs incubated in osteogenic media (OM), and gels with MSCs and BMP2 incubated in osteogenic media (OM+BMP2). One star indicates statistically significant difference (s.d.; p<0.05) between the test time point and first time point (day 4) in the same group and two stars indicates significant difference between the test group and all other groups at the same time point. Error bars correspond to means±1 SD for n=3. There was a significant difference in DNA content, ALPase activity and calcium content between the samples in BM and OM (p<0.05). There was not a significant difference in DNA content between the samples OM and OM+BMP2 (p=0.47). The ALPase activity and calcium content of the samples OM+BMP2 were significantly higher than OM.

FIG. 21 presents mRNA Expression levels, as fold difference, of DIx5 (FIG. 21A), Runx2 (FIG. 21B), OP (FIG. 21C), and OC (FIG. 21D) of MSCs encapsulated in SPELA-14.8 hydrogels. Experimental groups include gels with MSCs incubated in basal media (BM, left bar, control), gels with MSCs incubated in osteogenic media (OM, middle bar), and gels with MSCs and BMP2 incubated in osteogenic media (OM+BMP2, right bar). One star indicates statistically significant difference (s.d.; p<0.05) between the test time point and the first time point (day 4) in the same group and two stars indicates significant difference between the test group and all other groups at the same time point. Error bars correspond to means±1 SD for n=3. The OM+no MSCs group did not express any of the markers (no cell group). There was a significant difference in the expression of all markers between the samples in BM and OM (p<0.05). There was a significant difference in the expression of all markers between the samples in OM and OM+BMP2 (p<0.05).

### DETAILED DESCRIPTION

The following description and other modifications and variations to the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the following description is by way of example only, and is not intended to limit the invention.

Disclosed herein are hydrogel networks that are degradable and biologically inert. Beneficially, the degradation characteristics of the hydrogel networks can be controlled and the networks include a micelle-containing geometry in which potentially toxic materials can be sequestered within the core of the micelles. The degradable, in-situ gelling, biologically inert hydrogels with tunable properties are very attractive as a matrix for cell encapsulation and delivery, for instance to a site of regeneration.

The crosslinked hydrogel networks are based on a biocompatible polymer that is chain extended with a short hydrophobic segment and then crosslinked to form the micelles of the network. In development of the networks, it was recognized that degradation and crosslink density of previously known gels that incorporate hydroxy acids such as polylactic acid and viability of encapsulated cells is strongly dependent on the number of hydroxy acid monomers per macromonomer. The disclosed hydrogel networks take advantage of the recognition that macro ers with shorter lactide segments can produce mechanically robust hydrogels with tunable degradation rate.

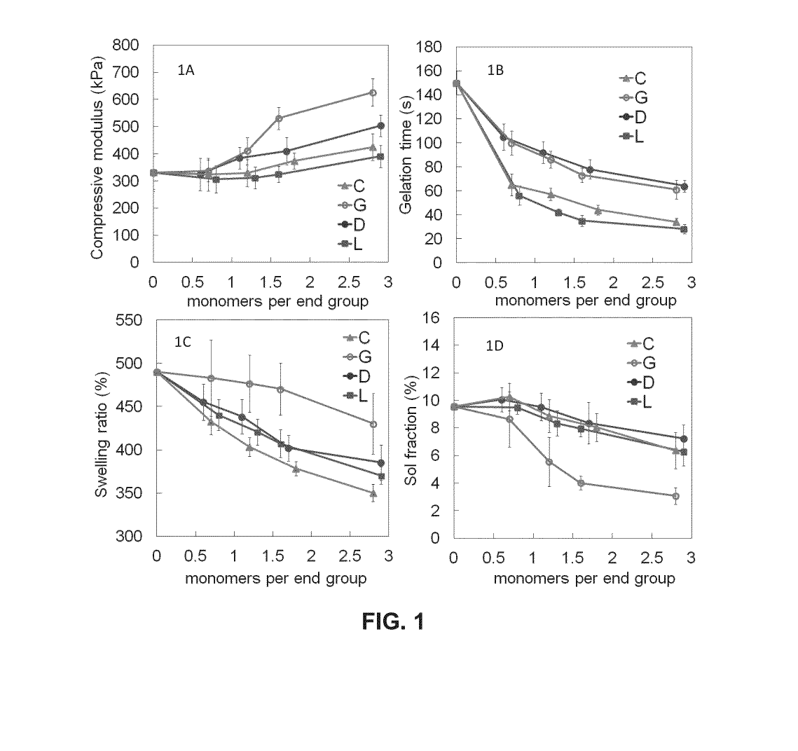

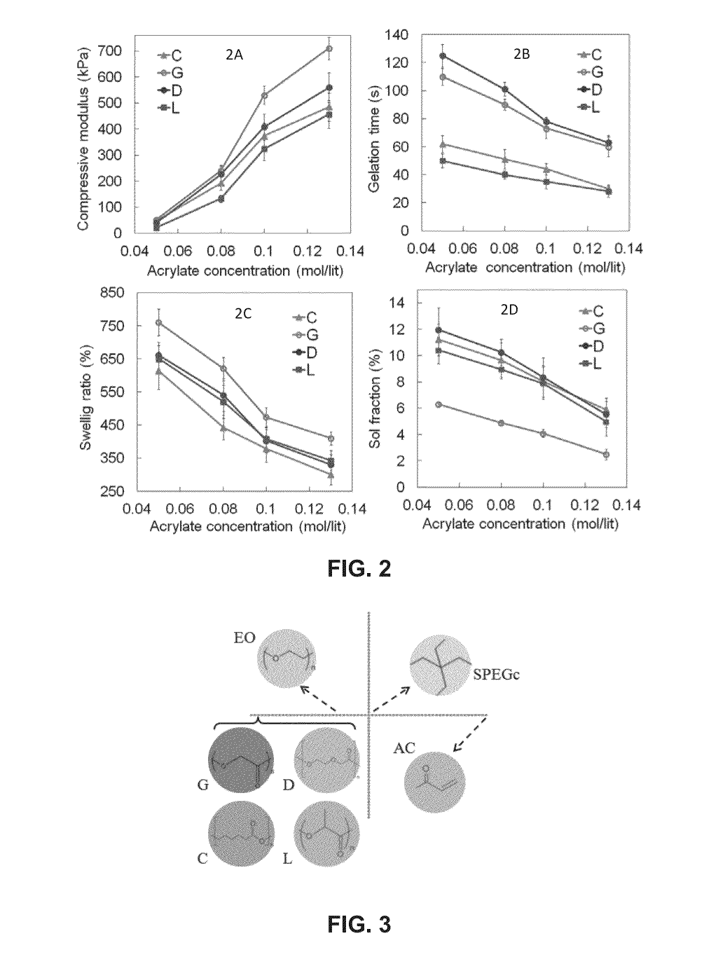

The crosslinked hydrogel networks can exhibit beneficial physical characteristics that can be controlled by the number of monomers included in the hydrophobic segment and/or by the concentration of the crosslinking moiety of the macromonomer. FIG. 1 graphically illustrates the effect of the number of monomers included in the hydrophobic segment on compressive modulus (FIG. 1A), gelation time (FIG. 1B), swelling ratio (FIG. 1C) and sol fraction (FIG. 1D) of crosslinked hydrogel networks formed with different monomers (lactide (L); glycolide (G), ε-caprolactone (C), or p-dioxanone (D)) in the chain extension, FIG. 2 graphically illustrates the effect of the concentration of crosslinking moiety (in this case an acrylate functionality) on compressive modulus (FIG. 2A), gelation time (FIG. 2B), swelling ratio (FIG. 2C) and sol fraction (FIG. 2D) of the same crosslinked hydrogel networks.

The crosslinked hydrogel networks can crosslink quickly, for instance with a gelation time of between about 20 seconds and about 180 seconds, or between about 25 seconds and about 150 seconds in some embodiments. As evidenced in the figures, the gelation time can decrease with increase in the number of hydrophobic monomers included in the hydrophobic segment as well as with an increase in the crosslinking moiety concentration.

The compressive modulus of the crosslinked hydrogel networks can generally be from about 50 kilopascals (kPa) to about 1000 kPa, or from about 200 kPa to about 600 kPa in some embodiments. The swelling ratio can be from about 250% to about 850%, or from about 350% to about 500% in some embodiments; and the sol fraction can be from about 2% to about 15%, or from about 2% to about 10% in some embodiments.

Polymers for use in forming the hydrogel network are not particularly limited and can include any biocompatible polymer as is generally known in the art for use in forming a biocompatible hydrogel. By way of example, biomedically useful synthetic polymers as have been utilized in the past including, without limitation, PEG, PVA, PHEMA, PVP, polyacrylic acid, polymethacrylates, polyacrylamide, polymethyl methacrylate, and the like as well as blends or copolymers can be utilized. The polymers are not limited to synthetic polymers, and in some embodiments natural polymers such as, without limitation, collagens, alginates, hyaluronic acids, cellulose and derivatives (e.g., carboxymethyl cellulose, hydroxyethyl cellulose), starches, chitosans, polysaccharides, and so forth can be utilized in forming the crosslinked hydrogel network. In addition, multiple different types of polymers may be incorporated in a crosslinked hydrogel network.

The molecular weight of the polymer is not particularly limited. For instance, the polymer can have a molecular weight range between about 1000 as and about 50,000 Da, though larger or smaller polymers can be utilized in other embodiments. In addition, the polymer can be branched or linear. For instance, the polymer can be a branched or star polymer having multiple polymer chains emanating from a central core group. In general, a branched polymer is considered a polymer having a limited number (e.g., three or four) different branches emanating from a central core, while a star polymer is considered a polymer having a large number (e.g., four or more) separate arms emanating from a central core. This is not a requirement, however, and the terms “branched polymer” and “star polymer” may be used interchangeably throughout this disclosure.

To form the hydrogel network, the biocompatible polymer is chain extended at the termini with short hydrophobic segments. More specifically, the hydrophobic segments can include no more than 5 hydrophobic monomers, or from 1 to 3 hydrophobic monomers in one embodiment.

The hydrophobic monomers can include any biocompatible hydrophobic monomers such as, without limitation, lipid monomers, anhydride monomers, orthoester monomers, phosphazene monomers, hydroxy acid monomers, and the like as well as mixtures of hydrophobic monomers. For instance, the hydrophobic segment can include no more than 5 hydroxy acid monomers such as, without limitation, glycolide, lactide, dioxanone, ε-caprolactone, hydroxy butyrate, valcrolactone, malonic acid, as well as mixtures of hydroxy acid monomers.

The short hydrophobic segments can be bonded to the polymer according to any suitable process, such as by combining the polymer with the hydrophobic monomer under reactive conditions with a catalyst, e.g., a tin(II)2-ethylhexanoate catalyst as described in detail in the Example section, below.

Without wishing to be bound to any particular theory, it is believed that the short hydrophobic segments can be sequestered within the core of the micelles formed during the crosslinking reactions. In addition, the crosslinking moieties and any initiators used in conjunction with the crosslinking reaction can be sequestered within the micellar core. By sequestering gelation and degradation reactions within the micellar structures that are formed upon the crosslinking of the macromonomers, those components of the hydrogel network that are involved in gelation and degradation, e.g., initiators, etc., can also be sequestered within the core of the micelles and this can reduce cytotoxicity of the network to cells that can be seeded on the network as well as to surrounding tissue.

Furthermore, the micelle size can be controlled by specific components used to form the network (e.g., the size and relative hydrophobicity of the segment), and the micelle size can directly affect the both the gelation and degradation rate of the crosslinked network. Thus, the degradation rate of the network can be tuned from a few days to many months depending on the specific materials utilized that in turn control the micelle size and equilibrium water content of the micelles, not the bulk equilibrium water content of the hydrogel.

Moreover, it has been found that these beneficial effects, e.g., sequestration of hydrophobic segments, crosslinking initiators, etc.; gelation rate control; degradation rate control; and so forth are only available when the hydrophobic segment that is bonded to the termini of the polymer is extremely short, i.e., no more than 5 hydrophobic monomers in length. By use of the short hydrophobic segments, micelles of from about 1 nanometer to about 5 nanometers are formed in the crosslinked network, and the micelles can sequester the hydrophobic components of the macromonomers.

Following the chain extension of the polymers with the hydrophobic segments, the macromonomer thus formed can be further processed to promote crosslinking of the macromonomer and formation of the crosslinked hydrogel network that includes the micelles. In one embodiment, the macromonomer can be acrylated at the termini and crosslinked via ultraviolet (UV) radiation according to standard practice. This is not a requirement, however, and any suitable crosslinking process can be utilized.

In some instances, crosslinking can occur through multiple functional groups at the termini of a branched or star polymer. For example, to crosslink the polymers via UV, the macromonomer can be functionalized at the termini to have UV a suitable functionality at the termini. Such groups are typically acrylates or methacrylates. The general scheme would include replacing terminal hydroxyl and/or carboxylic acid groups of the hydrophobic segment with acrylate or methacrylate functionality according to standard practice.

Crosslinking may be carried out via self-crosslinking of the macromonomer and/or through the inclusion of a separate crosslinking agents and/or initiators. Suitable crosslinking agents, for instance, may include polyglycidyl ethers, such as ethylene glycol diglycidyl ether and polyethylene glycol diglycidyl ether; acrylamides; compounds containing one or more hydrolyzable groups, such as alkoxy groups (e.g., methoxy, ethoxy and propoxy); alkoxyalkoxy groups (e.g., methoxyethoxy, ethoxyethoxy and methoxypropoxy); acyloxy groups (e.g., acetoxy and octanoyloxy); ketoxime groups (e.g., dimethylketoxime, methylketoxime and methylethylketoxime); alkenyloxy groups (e.g., vinyloxy, isopropenyloxy, and 1-ethyl-2-methylvinyloxy); amino groups (e.g., dimethylamino, diethylamino and butylamino); aminoxy groups (e.g., dimethylaminoxy and diethylaminoxy); and amide groups (e.g., N-methylacetamide and N-ethylacetamide).

If included, the initiator can be used to initiate crosslinking of the macromonomer. Examples of UV initiators include, without limitation, IRGACURE® 184 (1-hydroxycyclohexyl phenyl ketone), and DAROCURE® 1173 (α-hydroxy-1, α-dimethylacetophenone) which are both commercially available from Ciba-Geigy Corp, Additional examples of initiators (which may be UV-initiators, thermal initiators, or other types of initiators) may include, without limitation, benzoyl peroxide, azo-bis-isobutyronitrile, di-t-butyl peroxide, bromyl peroxide, cumyl peroxide, lauroyl peroxide, isopropyl percarbonate, methylethyl ketone peroxide, cyclohexane peroxide, t-butylhydroperoxide, di-t-amyl peroxide, dicymyl peroxide, t-butyl perbenzoate, benzoin alkyl ethers (such as benzoin, benzoin isopropyl ether, and benzoin isobutyl ether), benzophenones (such as benzophenone and methyl-o-benzoyl benzoate), acetophenones (such as acetophenone, trichloroacetophenone, 2,2-diethoxyacetophenone, p-t-butyltrichloro-acetophenone, 2,2-dimethoxy-2-phenylacetophenone, and p-dimethylaminoacetophenone), thioxanthones (such as xanthone, thioxanthone, 2-chlorothioxanthone, and 2-isopropyl thioxanthone), benzyl 2-ethyl anthraquinone, methylbenzoyl formate, 2-hydroxy-2-methyl-1-phenyl propane-1-one, 2-hydroxy-4′-isopropyl-2-methyl propiophenone, e-hydroxy ketone, tet-remethyl thiuram monosulfide, allyl diazonium salt, and a combination of camphorquinone or 4-(N,N-dimethylamino)benzoate.

Any of a variety of different crosslinking mechanisms may be employed, such as thermal initiation (e.g., condensation reactions, addition reactions, etc.), electromagnetic radiation, and so forth. Some suitable examples of electromagnetic radiation that may be used include, but are not limited to, electron beam radiation, natural and artificial radio isotopes (e.g., α, β, and γ rays), x-rays, neutron beams, positively-charged beams, laser beams, ultraviolet, etc. The wavelength λ of the radiation may vary for different types of radiation of the electromagnetic radiation spectrum, such as from about 10⁻¹⁴ meters to about 10⁻⁵ meters. Besides selecting the particular wavelength λ of the electromagnetic radiation, other parameters may also be selected to control the degree of crosslinking. For example, the dosage may range from about 0.1 megarads (Mrads) to about 10 Mrads, and in some embodiments, from about 1 Mrads to about 5 Mrads.

The crosslink integration numbers can increase with increasing numbers of hydrophobic monomers on the hydrophobic segment, which can result in a sharp decrease in gelation time. For instance, based on simulation results described in more detail in the Examples section, below, the crosslink moiety integration numbers increased with the number hydrophobic monomers per arm of a macromonomer resulting in a sharp decrease in gelation time of the precursor solutions. In addition, the number density of micelles and fraction of free polymer arms decreases as the number of hydrophobic monomers in each hydrophobic segment increases.

While not wishing to be bound to any particular theory, it is believed that the micelle formation changes the average crosslink distance (e.g., the average acrylate-acrylate distance), which affects mobility and reactivity of the crosslinking groups. In addition, it is believe that micelle formation and size can change the local concentration of hydrolytic groups (e.g., ester groups) and water throughout the crosslinked network, which can affect the hydrogel degradation rates and characteristics. Thus, by sequestering the hydrophobic components and the crosslinking components within micelles, the physical characteristics of the crosslinked hydrogel network can be finely tuned with regard to, e.g., degradation rates, compression modulus, sol percentage, gelation rates, and so forth.

The hydrolysis rate of the crosslinked hydrogel networks has been found through simulations to be strongly dependent on the hydrophobic monomer type and the number of hydrophobic monomers in the hydrophobic segment. For instance, a hydrogel network that incorporates a less hydrophobic monomer, such as glycolide, can degrade in a few days while one that incorporates a more hydrophobic monomer, such as ε-caprolactone, can degrade over the course of many months.

Furthermore, the effect of the number of hydrophobic monomers in the hydrophobic segment on hydrolysis rate of the crosslinked network can be bimodal. For example, as the number of glycolide monomers on each arm of a star PEG increases from 0.7 to 1.2, 1.8 and 2.8, mass loss after 2 days increased from 20% to 46 and 80% and then decreased to 66%, respectively. Similarly, as the number of lactide monomers on each arm of a star PEG increased from 0.8 to 1.7, 2.9 and 3.7, mass loss after 3 weeks increased from 32% to 50 and 62% and then decreased to 46%. The strong effect of hydrophobic monomer type and number on mass loss indicates that degradation of the hydrogels is controlled by equilibrium water content of the micelles, not bulk water content of the hydrogel. This understanding is strengthened by the fact that the initial water content of a crosslinked hydrogel network can be independent of hydrophobic monomer type and number.

The crosslinked hydrogel networks can support cell growth and differentiation for in vivo, ex vivo, or in vitro use. For example, a hydrogel can be loaded with one or more biologically active materials therein including cells, tissue explants, cellular extracts, and the like, which can be intended for growth and further proliferation within a system. Cellular extracts that may be incorporated into the hydrogels can include, but are not limited to, deoxyribonucleic acid (DNA), plasmids, ribonucleic acid (RNA), growth factors, lipids, suspect carcinogens, and suspect mutagens. Biological materials as can be incorporated in a hydrogel can be homogeneous from one single source, or from different sources. For instance, different cell types may be homogeneously distributed within a hydrogel.

Various techniques for isolating cells or tissues from suitable sources are generally known in the art, any of which can be utilized in conjunction with disclosed hydrogels. Moreover, cells or tissues can be autologous, allogenic, or xenogenic.

To promote the growth and differentiation of cells in a hydrogel, suitable signal molecules can be added to a culture medium, or to the hydrogel, to promote cell adhesion, growth, and migration. Examples of such signal molecules include, but are not limited to, serum, growth factors, and extracellular matrix proteins.

Cells can be genetically, physically or chemically modified prior or subsequent to being incorporated into a hydrogel. Genetic modification by molecular biology techniques is generally known in the art, any of which are encompassed herein. Methods are also known in the art to modify the immunological characters of allogenic or xenogenic cells. Immunologically inert cells, such as stem cells, infant cells, and embryonic cells can be used in conjunction with other cell types according to one embodiment, for instance to avoid immunological incompatibility.

A hydrogel may include biologically active compounds as may affect a developing system. For instance, a hydrogel can include a biologically active compound that can act as a signal for modifying cell adhesion, growth, or migration, preferably stimulating or promoting the adhesion, growth, or migration of the desirable cells, and/or inhibiting or stimulating the adhesion, growth, or migration of undesirable cells. Such compounds can include growth factors, hormones, extracellular matrix proteins and other cellular adhesion peptides identified in the extracellular matrix protein. Suitable growth factors may include, for example, epithelial growth factor (EGF), acidic or basic fibroblast growth factor (FGF), vascular endothelial growth factor (VEGF), hepatocyte growth factor (HGF), heparin binding growth factor (HGBF), transforming growth factor (TGF), nerve growth factor (NGF), muscle morphogenic factor (MMP), and platelet derived growth factor (PDGF). Examples of extracellular matrix proteins include fibronectin, collagens, laminins, and vitronectins, and the tri-peptide RGD (arginine-glycine-aspartate) that is found in many of the extracellular matrix proteins. A signal can also be included to induce the ingrowth of desirable cells, e.g., smooth muscle cells and epithelial cells. Compounds that inhibit or stimulate undesired cells, such as cancerous cells or inflammatory cells can be included.

The present disclosure may be better understood with reference to the Examples, set forth below.

### EXAMPLES

### Dissipative Particle Dynamic (DPD) Simulation Method

In DPD, each bead represents a soft particle interacting with the other beads via a soft pairwise force function given by:

$${f}_{ij} = {\sum }_{i \neq j}^{ } {F}_{ij}^{C} + {F}_{ij}^{D} + {F}_{ij}^{R} + {F}_{ij}^{S} \qquad ( 1 )$$

where fᵢⱼ is the total force and Fᵢⱼ^{C}, Fᵢⱼ^{D}, Fᵢⱼ^{R} and Fᵢⱼ^{S} are the conservative, dissipative, random and spring components of the force, respectively. Different components of the force in a cutoff distance (r_{c}) are calculated by

$$\begin{matrix}{F}_{ij}^{C} = \{ \begin{matrix}{α}_{ij} ( 1 - {r}_{ij} ) {e}_{ij} & \vert {r}_{ij} \vert < 1 \\ 0 & \vert {r}_{ij} \vert \geq 1\end{matrix} & ( 2 ) \\ {F}_{ij}^{D} = - γ [ {w}^{D} ( \vert {r}_{ij} \vert ) ] ( {e}_{ij} \cdot {v}_{ij} ) {e}_{ij} & ( 3 ) \\ {F}_{ij}^{R} = σ [ {w}^{R} ( \vert {r}_{ij} \vert ) ] {θ}_{ij} {e}_{ij} & ( 4 ) \\ {F}_{ij}^{S} = {\sum }_{j} {Cr}_{ij} & ( 5 )\end{matrix}$$

where rᵢⱼ is the vector joining bead i to j, eᵢⱼ and |rᵢⱼ| are the unit vector in the direction of rᵢⱼ and the magnitude of rᵢⱼ, respectively. vᵢⱼ is the velocity vector given by vᵢⱼ=vᵢ−vⱼ. w^{D} and w^{R} are the weight functions for dissipative and random forces, respectively, and γ, σ are the magnitude of dissipative and random forces. Fᵢⱼ^{D} and Fᵢⱼ^{R} act simultaneously to preserve the dissipation and to conserve the total momentum in the system. The dissipative and random force constants and weight functions are interrelated by w^{D}(rᵢⱼ)=[w^{R}(rᵢⱼ)]² and σ²=2k_{B}Tγ in order to satisfy the dissipation-fluctuation condition. The spring force term imposes geometrical constraints on the covalently bonded beads. The values of γ and C constants were 4.5 and 4, respectively. The repulsion between beads i and j is mainly dictated by the constant αᵢⱼ in the conservative force function. By choosing the system density ρ=3r_{c}⁻³, the DPD length scale, r_{c}, was 6.74 Å and the values of αᵢⱼ were determined using⁴⁰

αᵢⱼ=78+3.27χᵢⱼ (6)

where χᵢⱼ is the Flory-Huggins parameter between beads i and j. The values of χᵢⱼ in turn are given by:

$${χ}_{ij} = \frac{{( {δ}_{i} - {δ}_{j} )}^{2} V}{RT} \qquad ( 7 )$$

where δᵢ and δⱼ are the solubility parameters of beads i and j, respectively, V is the bead molar volume, T is absolute temperature, and R is the gas constant. The solubility parameters were calculated via atomistic molecular dynamics simulation performed via Forcite and Amorphous Cell modules, Materials Studio (v5.5, Accelrys) using the COMPASS force field, which is an ab initio force field optimized for condensed-phase systems. The position and velocity of the beads at each time point were obtained by solving the following equations of motion using the force function (equation 6).

$$\frac{d {r}_{i}}{d t} = {v}_{i} , \\ {m}_{i} \frac{d {v}_{i}}{d t} = {f}_{i} \qquad ( 8 )$$

All DPD simulations were performed in 30×30×30 r_{c} boxes with 3D periodic boundary conditions over 2×10⁵ time steps and dimensionless time step of 0.05. The Mesocite module of Materials Studio (v5.5, Accelrys) was used for DPD calculations.

### Materials

Lactide monomer (LA; >99, % purity) was purchased from Ortec (Easley, S.C.) and dried under vacuum at 40° C. for at least 12 h prior to use. Calcium hydride, tetrahydrofuran (THF), deuterated chloroform (99.8% deuterated), trimethylsilane (TMS), triethylamine (TEA), tin (II) 2-ethylhexanoate (TOO), acryloyl chloride, dimethylsulfoxide (DMSO), linear polyethylene glycol (LPEG, nominal Mw=4700), 4-arm PEG (SPEG, Mw=5000), ethylenediaminetetraacetic acid disodium salt (EDTA), penicillin, streptomycin, and paraformaldehyde were purchased from Sigma-Aldrich (St. Louis, Mo.). The protected amino acids and Rink Amide NovaGel resin for the synthesis of acrylamide-terminated GRGD peptide (SEQ ID NO: 9) were purchased from EMD Biosciences (San Diego, Calif.). Dichloromethane (DCM, Acros Organics, Pittsburgh, Pa.) was dried by distillation over calcium hydride. Diethyl ether and hexane were obtained from VWR (Bristol, Conn.). DCM Spectro/Por dialysis tube (molecular weight cutoff 3.5 kDa) was purchased from Spectrum Laboratories (Rancho Dominquez, Calif.). Dulbecco's phosphate-buffered saline (PBS) and Dulbecco's Modified Eagle's Medium (DMEM; 4.5 g/L glucose with L-glutamine and without sodium pyruvate) were obtained from GIBCO BRL (Grand Island, N.Y.). Trypsin and fetal bovine serum (FBS, screened for compatibility with rat BMS cells) were obtained from Invitrogen (Carlsbad, Calif.) and Atlas Biologicals (Fort Collins, Colo.), respectively. Quant-it PicoGreen dsDNA reagent kit was obtained from Invitrogen (Carlsbad, Calif.), QuantiChrom calcium and alkaline phosphatase (ALPase) assay kits were purchased from Bioassay Systems (Hayward, Calif.). BMP2 solution (100 μL, 1.5 mg/mL in BMP2 buffer) was generously donated by Medtronic (Minneapolis, Minn.). The Live/Dead calcein AM (cAM) and Ethidium homodimer-1 (EthD) cell viability/cytotoxicity kit was purchased from Molecular Probes (Life Technologies, Grand Island, N.Y.).

### Macromonomer Gelation and Rheological Measurements

The aqueous hydrogel precursor solution was crosslinked by UV free-radical polymerization using 4-(2-hydroxyethoxy)phenyl-(2-hydroxy-2-propyl) ketone (Irgacure 2959; CIBA, Tarrytown, N.Y.) photoinitiator as described, The initiator and macromonomer were dissolved separately in phosphate buffer saline (PBS; GIBCO BRL, Grand Island, N.Y.) by vortexing and heating to 50° C. The hydrogel precursor solution was prepared by mixing the macromonomer and initiator solutions and vortexing for 5 min. The crosslinking reaction was performed on a peltier plate of an AR-2000 rheometer (TA Instruments, New Castle, Del.) to monitor the gelation kinetics of the hydrogel precursor solution. A 20 mm plate acrylic geometry was used at a gap distance of 500 μm. A sinusoidal shear strain with frequency of 1 Hz and amplitude of 1% was exerted on the sample via the upper geometry. A long wavelength (365 nm) mercury UV lamp (Model B100-AP; UVP, Upland, Calif.) at a distance of 10 cm was used to irradiate the sample for up to 1000 s. The storage (G′) and loss moduli (G″) of the samples were recorded during gelation. The time at which G′=G″ was recorded as the gelation time.

### Measurement of Equilibrium Water Content

After crosslinking, samples with a diameter of 20 mm and thickness of 300 μm were removed from Peltier plate of the rheometer to measure water content. Samples were dried in ambient conditions for 12 h followed by drying in vacuum for 1 h at 40° C. Dry samples were swollen in DI water for 24 h at 37° C. with change of swelling medium every 6 h. After swelling, the surface water was removed and the swollen weights (wₛ) were measured. The swollen samples were dried as described above and dry weights (w_{d}) were recorded. The equilibrium water content was calculated by dividing the weight of water in the gel (the difference between swollen and dry weights) by swollen weight.

### Measurement of Mass Loss

The hydrogel precursor solutions were crosslinked in a PTFE mold (5 cm×3 cm×750 μm) covered with a transparent glass plate. Disc shape samples were cut from the gel using an 8 mm cork borer. The mass loss studies were performed in PBS (5 ml per sample) at 37° C. under mild agitation. At each time point samples were removed from the medium, washed with DI water several times and dried under vacuum. The dried sample weight was measured and compared with the initial dry weight to determine mass loss as described.

### Measurement of Swelling Ratio and Sol Fraction

After crosslinking, samples with 20 mm diameter×300 μm thickness were dried at ambient conditions for 12 h followed by drying in vacuum for 1 h at 40° C. and the dry weight (wᵢ) was recorded. Next, dry samples were swollen in DI water for 24 h at 37° C. and with a change of swelling medium every 6 h. After swelling, the surface water was removed and the swollen weight (wₛ) was recorded. Then, the swollen samples were dried and the dry weight (w_{d}) was recorded. The weight swelling ratio (Q) and sol fraction (S) were calculated by the following equations:

$$\begin{matrix}Q = \frac{{w}_{s} - {w}_{d}}{{w}_{d}} \times 100 & ( 9 ) \\ S = \frac{{w}_{i} - {w}_{d}}{{w}_{i}} \times 100 & ( 10 )\end{matrix}$$

### Marrow Stromal Cell Isolation and Encapsulation in the Hydrogel

MSCs were isolated from the bone marrow of young adult male Wistar rats as described. The bone marrow cell suspensions were centrifuged at 200 g for 5 min and cell pellets were resuspended in 12 mL basal medium which consisted of DMEM (GIBCO BRL, Grand Island, N.Y.) supplemented with 10% fetal bovine serum (FBS; Atlas Biologicals, Fort Collins, Colo.), 100 units/mL penicillin (PEN; Sigma-Aldrich), 100 μg/mL streptomycin (SP; Sigma-Aldrich), 50 μg/μL gentamicin sulfate (GS; Sigma-Aldrich), and 250 ng/mL fungizone (FZ; Sigma-Aldrich), and cultured in a humidified 5% CO2 incubator at 37° C. Cultures were replaced with fresh medium at 3 and 7 days to remove hematopoietic and other unattached cells. After 10 days, cells were detached from the flasks with 0.05% trypsin (Invitrogen, Carlsbad, Calif.)-0.53 mM EDTA (Sigma-Aldrich) and used for in vitro experiments.

The experimental groups for encapsulation of MSCs in hydrogels included m0, L-m1.7, D-m1.7, and C-m1.8. The cell encapsulation and osteogenic differentiation experiments were carried out in hydrogels chain extended with different hydroxy acid monomers while maintaining a constant compressive modulus of 50 kPa. Concentration of macromonomer in the precursor solution was varied to keep a constant compressive modulus. The hydrogel precursor solution was sterilized by filtration (0.2 μm average pore size). Acrylamide-terminated GRGD peptide (SEQ ID NO: 9) (Ac-GRGD (SEQ ID NO: 9)) was synthesized on Rink Amide NovaGel resin in the solid phase. The synthesized peptide was purified by high-performance liquid chromatography (HPLC) and characterized by electrospray ionization (ESI) mass spectrometry. The Ac-GRGD peptide (SEQ ID NO: 9) in the amount of 2 wt %, based on the macromonomer weight, was added to the hydrogel precursor solution to facilitate cell adhesion to the matrix. Next, 1×106 MSCs, suspended in 100 μL PBS, was gently mixed with the hydrogel precursor solution using a pre-sterilized glass rod. The final density of MSCs in the hydrogel was 5×106 cells/mL. The mixture was injected between two sterile microscope glass slides and cross-linked by UV irradiation as described above. The UV exposure time for all cell-seeded precursor solutions was 200 s, which was the minimum time for the gel modulus to reach its plateau value. After gelation, disk-shape samples ere incubated in 2 mL PBS for 1 h with two PBS changes. Next, the medium was replaced with complete osteogenic medium (basal medium supplemented with 100 nM dexamethasone (Dex), 50 μg/mL ascorbic acid (AA), 10 mM β-glycerophosphate (βGP)) and cultured for 28 days.

### Biochemical Analysis

At each time point (7, 14, 28 days), MSC encapsulated hydrogels were washed with serum-free DMEM for 8 h to remove serum components, washed with PBS, lysed with lysis buffer (10 mM tris and 2% triton), and sonicated to rupture the encapsulated cells. After centrifugation, the supernatant was used for measurement of total collagen content, alkaline phosphatase (ALP) activity and calcium content. Total collagen content of the samples was measured by a collagen assay kit (Sircol, Biocolor, Carrickfergus, UK) according to manufacturer's instructions. This method is based on selective binding of the G-X-Y amino acid sequence of collagen to Sircol dye. Briefly, 1 mL of Sircol dye was added to the sonicated cell lysate, incubated for 30 min and centrifuged at 10,000 rpm for 5 min to separate the collagen-dye complex. After removing supernatant, the collagen-dye complex was mixed with 1 mL Sircol alkali reagent and the absorbance was measured on a plate reader at 555 nm, ALP activity of the samples was measured using a ALP assay kit (QuantiChrom, Bioassay Systems, Hayward, Calif.) at 405 nm, Calcium content of the samples, as a measure of the total mineralized deposit, was measured using a Calcium Assay kit (QuantiChrom, Bioassay Systeme) at 575 nm.

mRNA Analysis

At each time point, total cellular RNA of the sample was extracted and converted to cDNA. The cDNA was amplified with gene specific primers designed using the Primer3 software. Expression of collagen type I (Col-I), Alkaline phosphatase (ALP) and Osteocalcin (OC) were measured by performing real-time quantitative polymerase chain reaction (RT-qPCR) using a CXF96 PCR system (Bio-Rad, Hercules, Calif.) using the following primers (synthesized by Integrated DNA technologies, Coralville, Iowa); Col-1: forward 5′-GCA TGT CTG GTT AGG AGA AAC C-3′ (SEQ ID NO:1) and reverse 5′-ATG TAT GCA ATG CTG TTC TTG C-3′ (SEQ ID NO:2); ALP: forward 5′CCT TGA AAA ATG CCC TGA AA-3′ (SEQ ID NO:3) and reverse 5′-CTT GGA GAG AGC CAC AAA GG-3 (SEQ ID NO:4); OC: forward 5′-AAA GCC CAG CGA CTC T-3′ (SEQ ID NO:5) and reverse 5′-CTA AAC GGT GGT GCC ATA GAT-3′ (SEQ ID NO:6); S16: forward 5′-AGT CTT CGG ACG CAA GAA AA-3′ (SEQ ID NO:7) and reverse 5′-AGC CAC CAG AGC TTT TGA GA-3′ (SEQ ID NO:8). The expression ratio of the gene of interest to that of S16 housekeeping gene was determined using PfaffI model and normalized to the first time point.

### Statistical Analysis

Data are expressed as means±standard deviation. All experiments were done in triplicate. Significant differences between groups were evaluated using a two-way ANOVA with replication test followed by a two-tailed Student's t-test. A value of p<0.05 was considered statistically significant.

### Example 1

Star polyethylene glycol (SPEC, 4 arm, nominal Mw=5 kDa, Sigma-Aldrich, St. Louis, Mo.) was chain-extended with short hydroxy acids (SPEX, X for hydroxy acid monomer) of X=lactide (L), glycolide (C), ε-caprolactone I, or p-dioxanone (D). The SPEG was synthesized by ring opening polymerization (ROP) according to known practice. The hydroxy acid monomers glycolide (G), lactide (L) and p-dioxanone (D) had >99.5% purity (Ortec, Easley, S.C.) and ε-Caprolactone monomer had >99% purity (Alfa Aesa, Ward Hill, Mass.). SPEC and tin (II) 2-ethylhexanoate (TOC, Sigma-Aldrich) were the polymerization initiator and catalyst, respectively. Briefly, the dry hydroxy acid monomer and SPEG were added to a three-neck reaction flask with an overhead stirrer and immersed in an oil bath (only SPEG was added to the flask for D polycondensation). The molar ratio of SPEG to monomer was selected based on the desired theoretical length of the hydroxy acid segment. Next, the reaction flask was heated to 120° C. under nitrogen stream to melt the mixture. After maintaining the temperature for 1 h to remove moisture, TOC catalyst was added to the mixture and the temperature was increased to the desired reaction temperature. For C and L monomers, the reaction was run at 140° C. for 12 h while for G monomer, the reaction was run at 160° C. for 10 h. Since the equilibrium is shifted toward monomer in polycondensation of p-dioxanone for temperatures >100° C., The SPEG and catalyst mixture was heated to 130° C. for 10 min to remove moisture, the mixture was cooled to 85° C., the dried D monomer was added, and the reaction was run at that temperature for 48 h. After the reaction, the product was purified by precipitation in ice cold hexane to remove any unreacted monomer, initiator and catalyst.

In the next step, the chain ends of the macromer were acrylated to produce the SPEXA macromonomer. The SPEX macromer (product of the first reaction) was dried by azeotropic distillation from toluene. Next, the macromer was dissolved in dichloromethane (DCM) and the reaction flask was immersed in an ice bath to control the temperature. The reaction was carried out by addition of equimolar amounts of acryloyl chloride (Ac, Sigma-Aldrich) and triethylamine (TEA, Sigma-Aldrich) drop-wise to the macromer solution under a dry nitrogen atmosphere. After 12 h, solvent was removed using rotary evaporation and the residue was dissolved in ethyl acetate to precipitate the byproduct triethylamine hydrochloride salt. After removing the ethyl acetate using vacuum distillation, the product was re-dissolved in DCM and precipitated in ice cold ethyl ether twice. Then, the product was dissolved in dimethylsulfoxide (DMSO) and dialyzed against water in a Spectro/Por dialysis tube (Spectrum Laboratories, Rancho Dominquez, Calif.; MW cutoff 3.5 kDa) to remove any remaining impurities. The SPEXA macromonomer was dried in vacuum to remove residual solvent and stored at −40° C.

Chemical structure of the synthesized product was characterized by a Varian Mercury-300 ¹H-NMR (Varian, Palo Alto, Calif.) at ambient conditions. The number- (Mₙ) and weight-average (M_{w}) molecular weight and polydispersity index (PI) of the macromonomer product were measured by Gel Permeation Chromatography (GPC, Waters 717 System, Milford, Mass.) in tetrahydrofuran (THF) with 1 mL/min flow rate.

Throughout this example, the notation X-mN is used for the hydrogels with X representing the hydroxy acid monomer, m for monomer, and N for the number of hydroxy acid monomers per macromonomer arm. For example, m0 denotes acrylated star PEG hydrogel without chain extension with hydroxyl acids, and C-m1.8 denotes SPECA hydrogel with average of 1.8ε-caprolactone monomers per macromonomer arm. The notation SPEXA-nA or SPEXA-mB are used to identify the length of the degradable segment, where A is the number of repeat units or ester groups per arm and B is the number of monomers per arm, and X is the monomer type (X=lactide (L), glycolide (G), ε-caprolactone I, or p-dioxanone (D)). When X is C or D, A equals B and when X is G or L, A=2B.

A solution of a polyethylene glycol/hydroxy acid/acrylate (SPEXA) macromonomer in water was simulated via DPD. The molecular structure of the macromonomer was divided into different beads with equal mass, as shown in FIG. 3. The beads included L (lactide repeat unit), G (glycolide), D (p-dioxanone), C (ε-caprolactone), EO (ethylene oxide repeat unit), Ac (acrylate functional group), SPEGc (star PEG core), and W (three water molecules). The meso-structure of the macromonomer is also shown in FIG. 3.

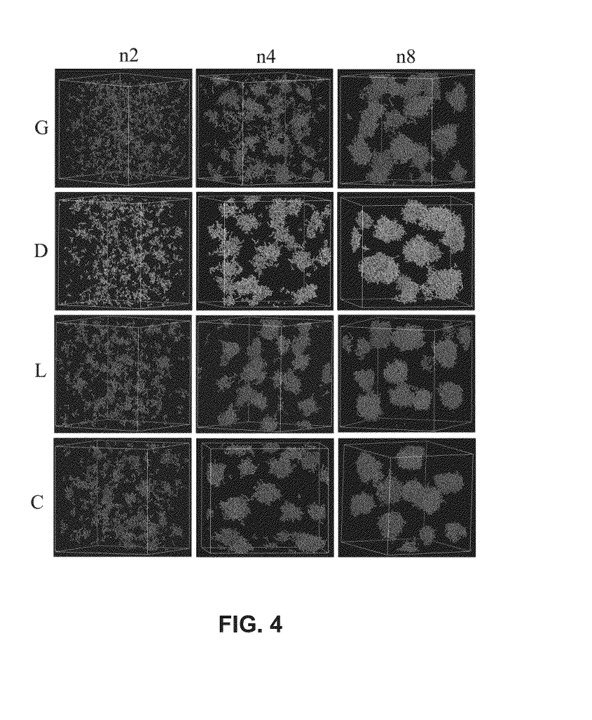

The formation of a nanoscale structure by SPEXA macromonomers in aqueous medium is shown in FIG. 4. In the absence of hydroxy acid monomers in the macromonomer, the distribution of acrylate groups attached to SPEXA chain ends was uniform in aqueous medium, but the extension of SPEXA arms with hydrophobic X segments initiated aggregation as shown in FIG. 4. For SPEGA and SPEDA with n=2, the hydrophobic segments were not sufficiently long to form stable micellar structures. However, SPELA and SPECA macromonomers (see first column of FIG. 4), due to the higher hydrophobicity of lactide and caprolactone monomers, formed stable micelles with n=2. All four SPEXA macromonomers formed stable micelles for n=4 (second column of FIG. 4). The aggregate size increased and number density decreased with increasing n from 4 to 8. According to the theory of micellization in block copolymers in solution, the degree of aggregation increases with increasing block size driven by the decrease in overall surface free energy of solvophobic blocks.

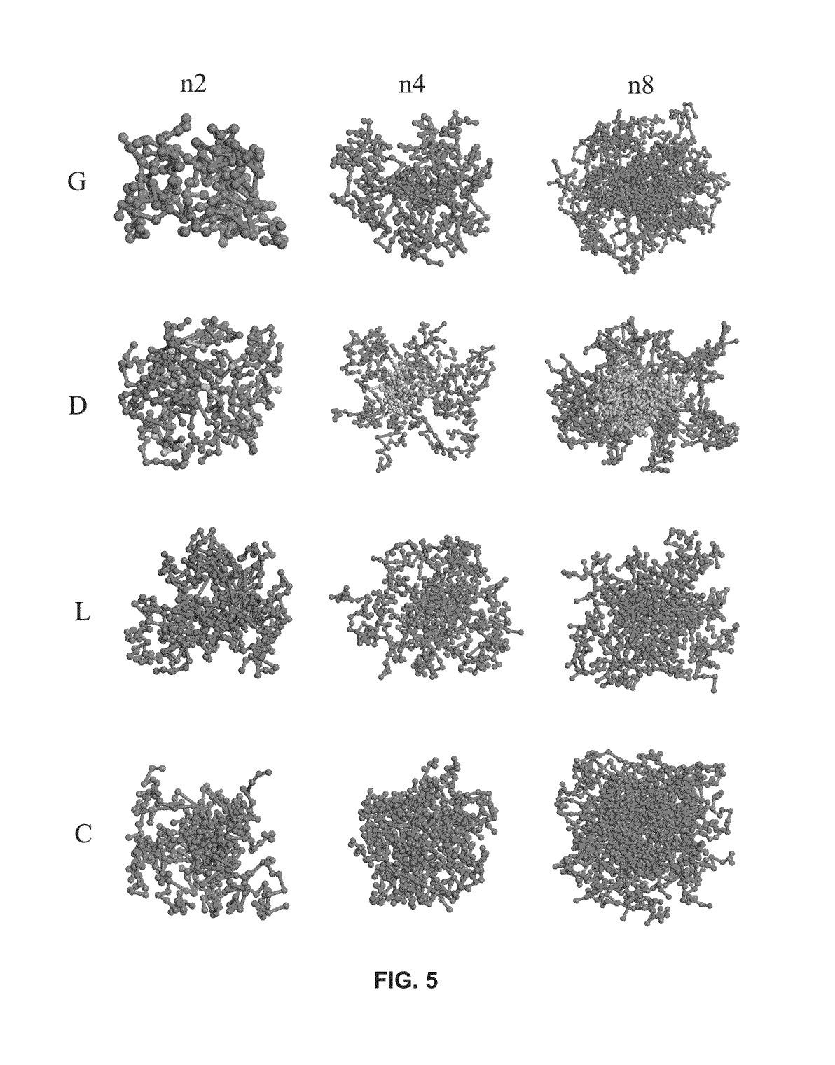

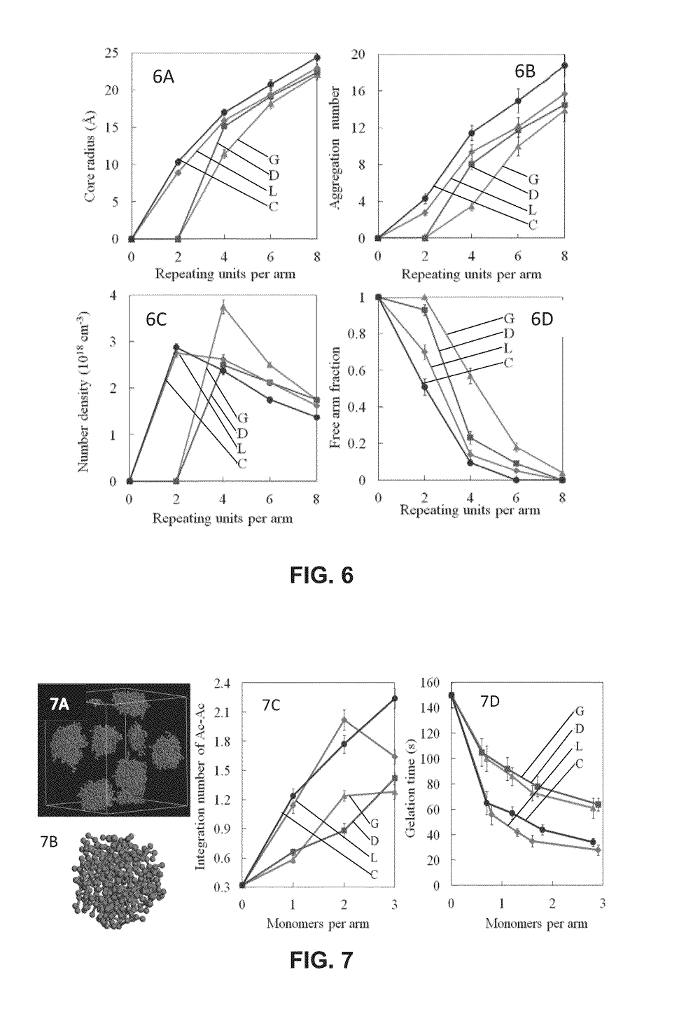

The cross-sectional view of an aggregate along with its EO beads is shown in FIG. 5 for different number of hydroxy acid monomers. In the images of FIG. 5, the hydrophobic hydroxy acid segments and hydrophilic EO beads formed the core and corona of the micelles, respectively. A change in core size and aggregation number of SPELA micelles is dominated by the interfacial free energy (the product of interfacial tension γ and interface area α). Due to the presence of hydrophilic EO segments at the interface, the effective interfacial tension in SPEXA micelles is different from the interfacial tension between the micelle core and water, γ_{C-W}. γ can be calculated by minimizing the chemical potential of the aqueous system at equilibrium:

$$γ = {γ}_{C - W} + \frac{kT}{{s}^{2}} [ ln \frac{1 - {C}_{i}}{1 - {C}_{b}} - \frac{N - 1}{N} ( {C}_{i} - {C}_{b} ) + {χ}_{W - BO} ( \frac{1}{2} {C}_{i}^{2} - \frac{3}{4} {C}_{b}^{2} ) ] \qquad ( 11 )$$

where γ_{C-W} is the interfacial tension between the micelles' core phase and water, k, T, s and N are the Boltzmann constant, absolute temperature, statistical length of the EO segment, and number of statistical EO segments on each SPEXA arm. Cᵢ and C_{b} are concentrations of EO segments at the interface and in the bulk, respectively. Equation 1 implies that an increase in Cᵢ has a negative contribution to the interfacial tension. In other words, dense EO coverage of the interface decreases the effective interfacial tension between the hydrophobic domains and water. According to FIG. 5, the micelle core size of SPECA macromonomers with n=8 was similar to that of the other monomers even though ε-caprolactone is significantly more hydrophobic than the other monomers. This discrepancy can be explained by the higher packing of the EO segments in the corona, thus decreasing the effective interfacial tension of the SPECA micelles. The effect of number of hydrophobic X beads on each macromonomer arm on core diameter of the micelles, number of macromonomers per micelle (aggregation number), number density of the micelles, and fraction of macromonomer free arms is shown in FIG. 6A, FIG. 6B, FIG. 6C, and FIG. 6D, respectively. Assuming there are only X and Ac beads in the core, the total number of beads taking part in core formation per macromonomer equals 4(n+1) and the aggregation number is calculated by:

$${n}_{agg} = \frac{ρ {V}_{c}}{4 ( n + 1 )} \qquad ( 12 )$$

where ρ and V_{c} are the bead number density in the system and micelle core volume, respectively. Core radius of the SPEGA and SPEDA micelles increased from 0 to 22 Å when n increased from 2 to 8. Core radius of the SPELA and SPECA micelles increased from 9 and 11 Å to 23 and 24 Å, respectively, with increasing n from 2 to 8. Aggregation number showed an increasing trend with n after micelle formation (n=2 for L and C and n=4 for G and D). The average aggregation number of SPECA increased from 4 to 19 when n increased from 2 to 8 which was the highest aggregation number among the four macromonomers. SPEGA had the lowest aggregation number, which ranged from 0 to 14 when n increased from 2 to 8. The increase in SPEGA aggregation number with increasing n is attributed to an increase in volume of the hydrophobic segments and a decrease in corona thickness of the micelles leading to an increase in the effective interfacial tension between the core and water with increasing n.

The number density of micelles initially increased with n due to the transition from uniform distribution of macromonomers in the system to the formation of micelles. The number density then decreased with n due to the increase in size and aggregation number of the micelles. FIG. 6D shows the effect of number of hydroxy acid monomers on each arm (n) on fraction of those macromonomer arms that are not part of the micelles (free arms). For n=0, the acrylates were uniformly distributed in solution and the fraction of free arms was unity. The fraction of free arms for SPELA decreased from 1 to 0.70, 0.14, 0.05 and 0 as n increased from 0 to 2, 4, 6 and 8, respectively while for SPEDA it decreased from 1 to 0.93, 0.23, 0.09 and 0. The fraction of free arms decreased at a faster rate for SPECA and reached 0 at n=6. SPEGA macromonomers with n≦2 did not form micelles and had a free arm fraction of unity, and the fraction of SPEGA free arms decreased to 0.57, 0.18 and 0.04 as n increased from 2 to 4, 6 and 8, respectively. The slower rate of decease in the fraction of free arms in SPEGA was consistent with the lower hydrophobicity of glycolide compared to other monomers.

The rate of crosslinking of SPEXA aqueous solutions depends on the proximity of acrylate groups to photo-activated acrylates while the rate of photo-activation of acrylates depends on the proximity of initiator molecules to acrylate groups. Therefore, the rate of crosslinking of SPEXA solutions depends on the average distance between the acrylates and initiator molecules. The distribution of photoinitiator beads in SPELA-m3 solution and cross-section of one of the micelle cores are shown in FIG. 7A and FIG. 7B, respectively. Simulation images indicate that 98% of the photoinitiator beads partitioned to the hydrophobic core of the micelles in the vicinity of acrylates. Therefore, formation of aggregates dramatically reduced the average inter-acrylate and acrylate-initiator separation distances, leading to a significant increase in the rate of initiation and propagation of the acrylates. To quantify the average inter-acrylate distance (related to crosslinking rate) or the proximity of water beads to ester groups on SPEXA macromonomers (related to the rate of hydrolytic degradation), the average number of Ac (or W) beads in a sphere of radius R around an Ac (or ester) bead or the running integration number of beads “a” around beads “b”, IN_{ab}(R) was calculated by:

$${IN}_{ab} ( R ) = 4 {πρ}_{b 0} {\int }_{0}^{R} {g}_{ab} ( r ) {r}^{2} d r \qquad ( 13 )$$

where ρ_{b0} is the overall number density of type “b” beads and g_{ab}(r) is the radial distribution function of bead “b” around bead “a”, located at the origin. The running integration number of Ac-Ac beads in SPELA solutions (IN_{Ac-Ac}) at R=r_{c} (6.74 Å, the DPD length scale, see Methods section) first increased with increasing m from 0 to 2, as shown in FIG. 7C, and then decreased as m increased from 2 to 3. Conversely, a unimodal increase in IN_{Ac-Ac} was observed for SPEGA, SPECA and SPEDA solutions with increasing m from 0 to 3. The increase in IN_{Ac-Ac} with m is attributed to a decrease in the average Ac-Ac distance in the core of the micelles. As the core of the micelles continued to increase in size and their separation distance continued to increase for m>2, the average distance between the Ac beads began to increase, leading to a decrease in IN_{Ac-Ac}, as predicted for SPELA in FIG. 7C. Furthermore, SPELA and SPECA macromonomers had higher IN_{Ac-Ac} than SPEGA and SPEDA for m≦3. The predicted IN_{Ac-Ac} values are related to gelation time of the macromonomers in aqueous solution. The gelation time of the SPEXA solutions with 20 wt % macromonomer was measured by rheometry as a function of number of hydroxy acid monomers per arm and the results are shown in FIG. 7D. The gelation times of SPELA and SPECA solutions were shorter than those of SPEGA and SPEDA as predicted by simulation (see FIG. 7C). The gelation time of SPEGA, SPEDA, SPELA and SPECA solutions (20 wt % concentration) decreased from 150 s to 61, 64, 28 and 34 s, respectively, with increasing m from 0 to 3 (see FIG. 7D). The initial dramatic decrease in gelation time is attributed to aggregate formation and a sharp increase in IN_{Ac-Ac}. Simulation results in FIG. 5c predict that the IN_{Ac-Ac} value for SPELA decrease for m>2. However, a decrease in IN_{Ac-Ac} for SPELA at higher m values was offset by an increase in residence time of the arms in the micelle core, leading to no increase in gelation time. The residence time of a hydrophobic segment in the micelle core is proportional to:

τ˜γ·n^{2/3} (14)

where γ is the effective interfacial tension between the hydrophobic domains and water. As a result, residence time of the unreacted Ac groups in the core of micelles increased with n which increased the rate of crosslinking, thus reducing gelation time. Furthermore, fraction of bridging arms between micelles increased with increasing residence time of the arms which in turn increased the extent of physical gelation. Therefore, gelation time of the macromonomer solution continued to decrease with increasing n.

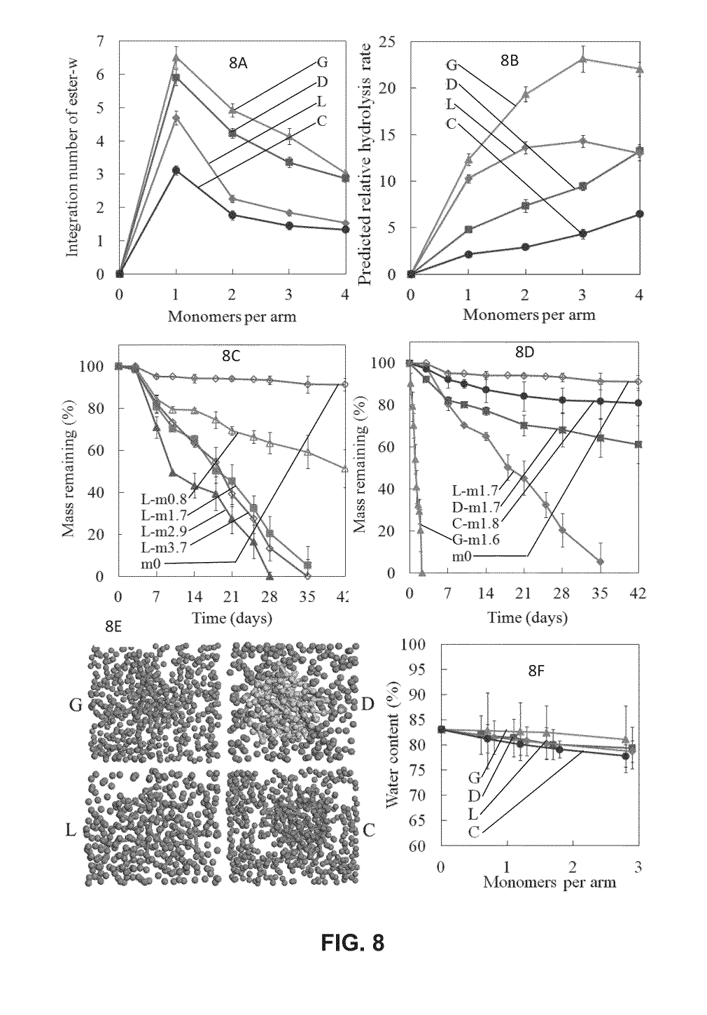

The degradation rate of SPEXA hydrogels depends on the proximity of water beads to the ester links on short hydroxy acid segments on each arm of SPEXA macromonomer. The local distribution of water beads around hydrophobic cores of SPEXA-n4 micelles is shown in FIG. 8E. The water beads were in close proximity to G beads in SPEGA solution. The relatively small size of the G cores along with the lower hydrophobicity of G beads compared to other hydroxy acids led to a short average distance between the G and W beads. The size of the core increased and local concentration of W beads around the core decreased when SPEGA was replaced with SPELA solution, as shown in FIG. 8E (see image “L”), thus increasing the average distance between L and W beads. A dip in the concentration of W beads observed proximal to the micelle core in SPELA is attributed to the higher hydrophobicity of L beads, compared to G, leading to a higher packing of EO beads at the interface of the core with aqueous medium. The concentration of W beads proximal to the core increased by changing SPELA with SPEDA solution but core size in SPEDA was significantly larger than that of SPEGA. The lowest concentration of W beads at the core margins was observed for SPECA micelles where the high hydrophobicity of C cores overcame the energy of chain extension and forced the EO beads to undergo high-entropy packing at the interface of the core and aqueous medium by repelling water beads from the interfacial layer.

The effect of the number of monomers per arm (m) on running integration number of water beads around ester links, IN_{ester-W}, for SPEXA macromonomers is shown in FIG. 8F. IN_{ester-W} initially increased with the addition of one monomer to SPEXA and subsequent aggregation of short hydroxy acid segments. Then, due to the increase in micelle size and decrease in total micelle surface area, IN_{ester-W} decreased for all SPEXA solutions with m>1. Due to the lowest and highest hydrophobicity of glycolide and ε-caprolactone monomers, SPEGA and SPECA solutions had the highest and lowest IN_{ester-W} for all m values, However, degradation rate of SPEXA hydrogel depends on the density of ester groups in the hydrogel volume as well as the proximity of ester groups to water beads, Assuming that carboxylic acid formation by dissociation of esters does not affect the hydrolysis rate constant (this is a good assumption since degradation is performed in a buffered aqueous medium), the relative hydrolysis rate (P) at the simulation scale (the mesoscale) which is proportional to the macroscale rate of degradation is defined as:

P=IN_{ester-W}INₑₛₜₑᵣ₋ₑₛₜₑᵣ (15)

In the above equation, IN_{ester-W} and INₑₛₜₑᵣ₋ₑₛₜₑᵣ are proportional to the concentration of water and ester groups in the micelles, respectively. The simulated relative hydrolysis rate in the reaction volume for SPEXA macromonomers (20 wt %) in aqueous solution as a function of m is shown in FIG. 8B. For all m values, SPEGA had the highest relative hydrolysis rate followed by SPELA, SPEDA, and SPECA. The relative hydrolysis rate for SPECA and SPEDA solutions increased from zero to 5.2 and 12.5, respectively, with increasing m from zero to 4. Likewise, the relative hydrolysis rate of SPELA and SPEGA solutions increased from zero to 13.4 and 22.5, respectively, with increasing m from zero to 3 and then it decreased to 12.3 and 21.0 with increasing m from 3 to 4. The relatively large difference in predicted relative hydrolysis rates between SPEXA macromonomers at a given m indicates that hydrolysis in these systems is related to the equilibrium water content and concentration of ester groups in the micelles, not to bulk concentrations (the solutions had similar bulk water and SPEXA contents). The predicted bimodal hydrolysis rate for SPELA and SPEGA in FIG. 8B is attributed to the low proximity of water to ester beads in larger micelle cores at higher m values. SPECA with the most hydrophobic (lowest water content) micelles had the lowest predicted hydrolysis rate while SPEGA with the least hydrophobic (highest water content) micelles had the highest hydrolysis rate. To compare with predictions, mass loss of SPELA hydrogels (20 wt %) with incubation time for different m values is shown in FIG. 8C. SPELA-0L without lactide chain extension had <5% mass loss after 6 weeks of incubation. Mass loss of SPELA gels was linear with incubation time for all m values. SPELA hydrogels lost 6%, 37%, 80%, and 100% mass after 4 weeks as m increased from zero, 0.8, 1.6, and 2.9, respectively. However, SPELA mass loss decreased from 100% to 87% as m was increased from 2.9 to 3.7, which was consistent with the predicted decrease in SPELA hydrolysis rate for 3≦m≦4 in FIG. 8B. The experimentally measured mass loss of SPEXA hydrogels at similar m values (1.6≦m≦1.8) is compared in FIG. 8D. In comparing the mass loss of SPEXA hydrogels with average m value of 1.7, SPEGA completely degraded in 3 days, SPELA completely degraded at a constant rate in 5 weeks, SPEDA and SPECA lost 40% and 20% mass in 6 weeks, respectively. Equilibrium water content of SPEXA hydrogels as a function of m is shown in FIG. 8F. The difference in water content of SPEXA gels with different monomers was not statistically significant (p values for the difference between the water contents of G and D, G and L, and G and C were >0.17). Therefore, the wide range of degradation rates from a few days to many months observed for SPEXA gels with different hydroxy acids, as shown in FIG. 8D, are not due to differences in water content. The differences can only be explained by large variations in equilibrium water content of the micelles as a function of hydroxy acid type.

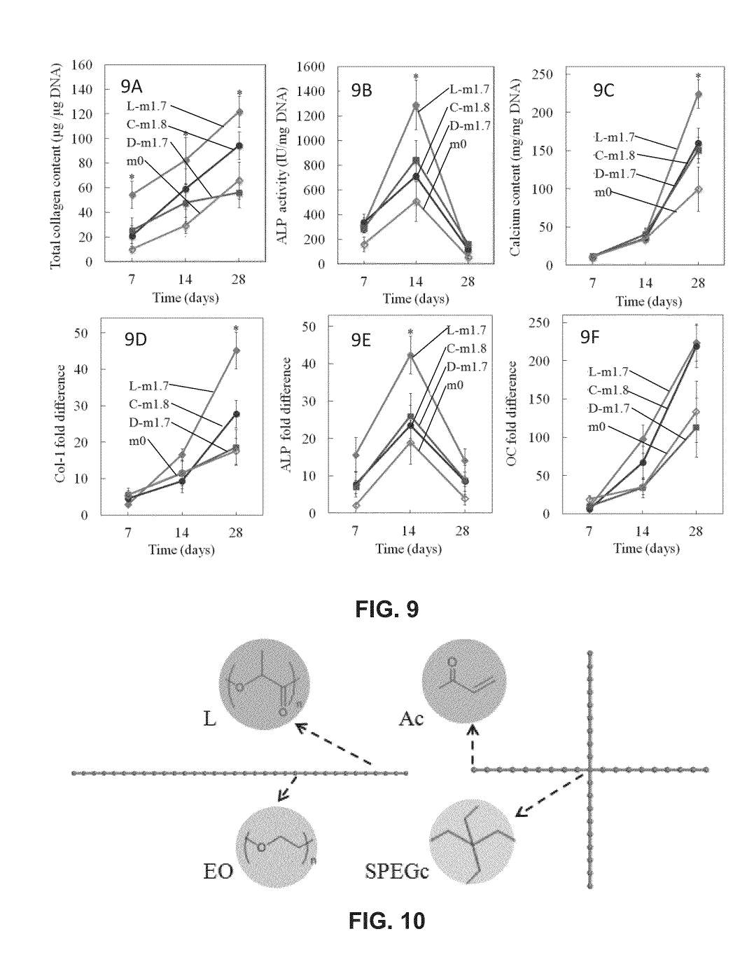

Multipotent stromal cells (MSCs) were encapsulated in SPEXA hydrogels and the effect of macromonomer type on differentiation of MSCs was evaluated by incubation in osteogenic medium. SPEGA gel due to its fast mass loss and degradation (see FIG. 8D) was not used for MSC encapsulation. Groups included 20 wt % SPELA (L-m1.7), SPEDA (D-m1.7), SPECA (C-m.18), and PEG (m0). The effect of hydroxy acid type on total collagen content with incubation in osteogenic medium is shown in FIG. 9A. Total collagen content of L-m1.7 gels increased from 54 μg/μg DNA at day 7 to 83 and 122 μg/μg DNA at days 14 and 21, respectively. Total collagen content of L-m1.7 gels was significantly higher than that of C-m1.8, D-m1.7 and m0 gels for all incubation times. The higher secretion of collagen by MSCs seeded in L-m1.7 gels was attributed to the higher degradation rate of SPELA gel compared to other gels. It is well established that the rate of ECM production and ECM quality is related to the rate of matrix degradation. With slow degradation, there is limited pore volume for tissue growth and the produced ECM is localized to the pericellular region of the cells. With fast degradation, rate of matrix degradation surpasses ECM production resulting in disintegration of the matrix with incubation time and lower extent of cell adhesion.

ALP activity and extent of mineralization of MSCs encapsulated in SPEXA hydrogels are shown in FIG. 9B and FIG. 9C, respectively. Consistent with previous results. ALP activity of all groups increased from day 7 to 14 and then decreased at day 28 with the start of mineralization. At day 14, ALP activity of L-m1.7 was significantly higher than other groups indicating higher osteogenic differentiation of MSCs in SPELA gel. Calcium content of all four groups had an increasing trend with time. Calcium content of L-m1.7 increased from 10.3±1.2 to 40.8±8.5 and 224.7±18.5 mg/mg DNA with incubation time from day 7 to 14 and 28. At day 28, the calcium content of L-m1.7 was significantly higher than other groups. Furthermore, at day 28, calcium content of D-m1.7 and C-m1.8 was significantly higher than that of m0 group. It can be inferred from the results that degradation of SPEXA gels contributed significantly to mineralization, In addition, L-m1.7 hydrogel with the highest degradation rate showed 2.3 times higher mineral deposition by MSCs compared to non-degradable m0 group (PEG gel). The higher extent of mineralization of MSCs in degradable gels was consistent with previous reports. For example, human MSCs encapsulated in a hydrolytically degradable PEG matrix and incubated in osteogenic medium displayed higher cell-cell contact and cell spreading compared to non-degradable PEG matrix. In another study, mineralization by osteoblasts increased three folds with incorporation of degradable lactide segments in PEG dimethacrylate hydrogels.

mRNA expression levels of Col-1, ALP and OC are shown in FIG. 9D-FIG. 9F, respectively. Col-1 expression for all groups was significantly higher at day 28 compared to day 7. For example, Col-1 mRNA expression of L-m1.7 increased from 2.9±0.7 to 16.6±1.7 and 45.2-15.0 when incubation time increased from day 7 to 14 and 28. At day 28, Col-1 mRNA expression of L-m1.7 was 2.5 fold higher than that of non-degradable m0 gel. ALP mRNA expression (FIG. 9E) showed a trend similar to its bioactivity in FIG. 9B. At day 14, ALP mRNA expression of L-m1.7 was significantly higher than other gels and 2.2 times higher than the non-degradable m0 gel. OC expression of all groups increased from day 7 to day 28 with L-m1.7 and C-m1.8 gels showing significantly higher OC expression at day 28 compared to D-m1.7 and m0 gels. Taken together, the findings of this work demonstrate that chain extension of PEG hydrogels with short hydroxy acid segments results in the formation of micellar hydrogels with a wide range of degradation rates from a few days to a few months that support differentiation and mineralization of marrow stromal cells.

### Example 2

A two-step procedure was used to synthesize linear (LPELA) and star (SPELA) poly(ethylene glycol-co-lactide) acrylate macromonomers. In the first step, linear (LPEL) and star (SPEL) poly(ethylene glycol-co-lactide) macromers were synthesized by melt ring-opening polymerization of lactide with LPEG and SPEG, respectively as polymerization initiators and with TOC as the reaction catalyst. LPEG and SPEG were dried by azeotropic distillation from toluene prior to the reaction. The LA and PEG were added to a three-neck reaction flask equipped with an overhead stirrer. The LA:PEG molar ratio was varied from 0 to 20 to synthesize macromonomers with different lactide segment lengths. The reaction flask was heated to 120° C. with an oil bath under steady flow of dry nitrogen to melt the reactants. Next, 1 ml of TOC was added and the reaction was allowed to continue for 8 h at 135° C. After the reaction, the product was dissolved in DCM and precipitated in ice cold methanol followed by ether and hexane to fractionate and remove the unreacted monomer and initiator. The synthesized LPEL and SPEL macromers were vacuum dried to remove any residual solvent and stored at −20° C.

In the next step, the terminal hydroxyl groups of LPEL and SPEL macromers were reacted with acryloyl chloride to produce LPELA and SPELA macromonomers, respectively. Prior to the reaction, macromers were dissolved in DCM and dried by azeotropic distillation from toluene to remove residual moisture. After cooling under steady flow of nitrogen, the macromer was dissolved in DCM and the reaction flask was immersed in an ice bath. Equimolar amounts of acryloyl chloride and TEA were added drop-wise to the solution to limit the temperature rise of the exothermic reaction. The reaction was allowed to proceed for 12 h. After the reaction, solvent was removed by rotary evaporation and the residue was dissolved in ethyl acetate to precipitate the by-product triethylamine hydrochloride salt. Next, ethyl acetate was removed by vacuum distillation and the macromer was re-dissolved in DCM and precipitated twice in ice cold ethyl ether. The synthesized macromonomer was dissolved in DMSO and purified by dialysis to remove any unreacted acrylic acid. The LPELA and SPELA products were dried in vacuum to remove residual solvent and stored at −40° C. The chemical structure of the macromonomers was characterized by a Varian Mercury-300 H-NMR (Varian, Palo Alto, Calif.) at ambient conditions with a resolution of 0.17 Hz.

The notations LPELA-nLa-Mb and SPELA-nLa-Mb are used to identify the architecture (linear versus star) as well as composition of the samples, where a and b represent number of lactide monomers (nL) per macromonomer and macromonomer concentration (wt %), respectively.

The aqueous solutions of SPELA and LPELA macromonomers were simulated via DPD approach. FIG. 10 shows the molecular structure and different bead types on SPELA and LPELA macromonomers. The bead types with equal mass and volume in the simulation volume were L (one lactide monomer), EO (four ethylene oxide repeat units), Ac (the acrylate group), SPEGc (the star PEG center) and W (eight water molecules). In DPD, each bead represents a soft particle interacting with the other beads via a soft pair-wise force function given by Equation (1), as described previously. In this simulation, the system density ρ=3r_{c}⁻³, the DPD length scale, r_{c}, was 8.18 Å. Fᵢⱼ^{D}, Fᵢⱼ^{R} and Rᵢⱼ^{S} were calculated as previously described. Fᵢⱼ^{C} was calculated from the pair-wise interaction parameter between beads i and j, αᵢⱼ. The αᵢⱼ were determined using the Flory-Huggins parameter between beads i and j, χᵢⱼ, by the following equation:

αᵢⱼ=25+3.27χᵢⱼ (16)

The Flory-Huggins parameters were calculated via atomistic molecular dynamics simulation (Forcite and Amorphous Cell modules, Materials Studio v5.5, Accelrys) using the COMPASS force field, which is an ab initio force field optimized for condensed-phase systems. All DPD simulations were performed in a 30×30×30 r_{c} simulation box with 3D periodic boundary conditions with over 200000 time steps and dimensionless time step of 0.05. The Mesocite module of the Materials Studio (v5.5) was used to perform the DPD calculations.

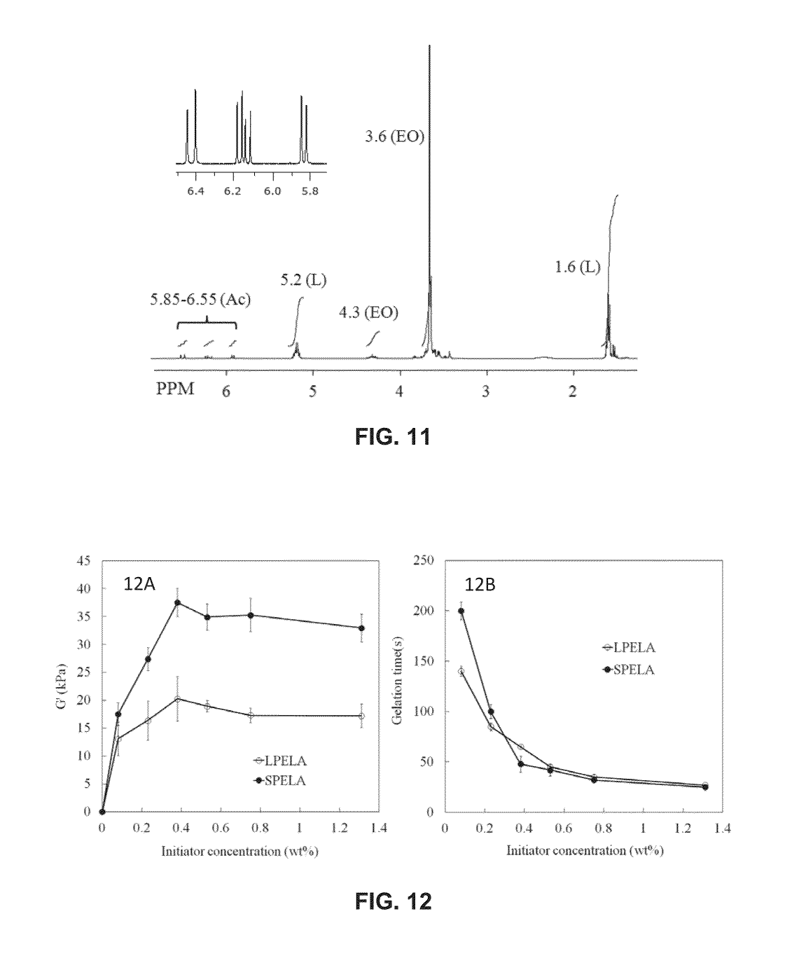

¹H-NMR spectrum of SPELA-nL14.8 macromonomer is shown in FIG. 11. The chemical shifts with peak positions at 3.6 and 4.3 ppm were attributed to the methylene hydrogens (═CH₂) of PEG attached to ether (—CH₂—O—CH₂—) and ester (—CH₂—OOC—) groups of lactide respectively. The shifts with peak positions at 1.6 and 5.2 ppm were attributed to the methyl (—CH₃) and methine (═CH) groups of lactide respectively. The shifts with peak positions from 5.85 to 6.55 ppm (see inset in FIG. 11) were attributed to the vinyl hydrogens (—CH═CH₂) of the acrylate group at the end of each macromonomer arm as follows: Peak positions in the 5.82-5.87 ppm range were associated with the trans proton of unsubstituted carbon of the Ac: those in the 6.10-6.20 ppm range corresponded to the protons bonded to monosubstituted carbon of the Ac; and those in the 6.40-6.46 ppm range were associated with the proton of unsubstituted carbon of the acrylate group. The M̅ₙ̅ of SPELA was determined from the ratio of shifts centered at 1.6 and 5.2 ppm (lactide hydrogens) to those at 3.6 and 4.3 ppm (PEG hydrogens), The number of acrylate groups per macromonomer was determined from the ratio of shifts between 5.85 and 6.55 ppm (acrylate hydrogens) to those at 3.6 and 4.2 ppm (PEG hydrogens). The average number of lactide monomers (nL), average acrylate groups per macromonomer, and M̅ₙ̅ for LPELA and SPELA macromonomers as a function of lactide to PEG (LEGF) molar feed ratio are summarized in Table 1.

TABLE 1

| | | Lactides per | Average | Average |
|---|---|---|---|---|
| | | end group | number of | number of |
| | | (macro- | lactides | acrylates |
| Macro- | M̅ₙ | monomer) | per end | per end |
| monomer | (±100) | in the feed | group (±0.1) | group (±0.05) |
| SPELA-nL0 | 5300 | 0 | 0 | 0.85 |
| SPELA-nL3.4 | 5800 | 1.25 (5) | 0.8 | 0.86 |
| SPELA-nL6.4 | 6200 | 2.5 (10) | 1.6 | 0.82 |
| SPELA-nL11.6 | 6900 | 3.75 (15) | 2.9 | 0.74 |
| SPELA-nL14.8 | 7400 | 5 (20) | 3.7 | 0.75 |
| LPELA-nL0 | 4700 | 0 | 0 | 0.89 |
| LPELA-nL3.6 | 5200 | 2.5 (5) | 1.8 | 0.85 |
| LPELA-nL7.4 | 5800 | 5 (10) | 3.7 | 0.87 |
| LPELA-nL9.6 | 6100 | 7.5 (15) | 4.8 | 0.77 |
| LPELA-nL14.8 | 6800 | 10 (20) | 7.4 | 0.71 |

LEGF ratio was varied from zero to 20 with intervals of 5, as shown in column 3 of the table. The nL values, shown in the first column, changed from 0 to 3.4, 6.4, 11.6 and 14.8 for SPELA and from 0 to 3.6, 7.4, 9.6 and 14.8 for LPELA as LEGF values were increased from zero to 5, 10, 15, and 20, respectively. As LEGF ratio was increased from zero to 20. M̅ₙ̅ of SPELA (column 2) increased from 5.2 to 7.4 kDa and M̅ₙ̅ of LPELA increased from 4.7 to 6.8 kDa. As LEGF ratio was increased from zero to 20, number of lactides per arm of the macromonomer (column 4) increased from zero to 3.7 for SPELA and from zero to 7.4 for LPELA. The range for the average number of acrylate groups per arm of SPELA and LPELA macromonomers was 0.75-0.86 and 0.71-0.89, respectively. It should be noted that the standard deviation from the mean (s.d.) for M̅ₙ̅, average number of lactide units per end group, and average number of acrylates per end group were 100 Da, 0.1, and 0.05, respectively. Therefore, the differences in acrylate groups per end group for SPELA-nL0, SPELA-nL3.4, and SPELA-nL6.4 (0.85, 0.86, and 0.82, respectively) and those between LPELA-nL0, LPELA-3.6 nL, and LPELA-7.4 nL (0.89, 0.85, and 0.87, respectively) were not statistically significant. In general, there was a decrease in the average number of acrylates per arm with increasing nL. This decrease was related to higher steric hindrance of hydroxyl end-groups in LPELA and SPELA macromonomers with longer length of lactide segments, leading to a lower effective reactivity with acryloyl chloride. In general, polydispersity of SPELA and LPELA macromonomers was <1.5.

The intersection of storage and loss moduli (G″), where G′=G″, was used to determine the gelation time. All time sweep tests exhibited a lag or induction time, a developing portion, and a plateau region. However the length of each region as well as the final value of G′ were affected by the macromonomer structure, i.e., linear versus star and number of lactides. In general, the time for induction/lag time decreased with increasing nL, because the average distance between the reactive acrylate groups decreased with increasing nL. The slope and duration of the developing portion of the gelation curve decreased with increasing nL. There was also a decrease in plateau shear modulus with increasing nL. The minimum UV exposure time for the gels to reach their plateau modulus was 600 sec.

The effect of initiator concentration on the storage modulus and gelation time of LPELA-nL7.4 and SPELA-nL14.8 macromonomers (both having 3.7 lactide monomers per end group) is shown in FIG. 12A and FIG. 12B, respectively. SPELA-nL14.8 has the longest hydrophobic lactide segment length, compared to other SPELA macromonomers, leading potentially to highest steric hindrance of the acrylate groups in each arm in aqueous solution, and lowest effective reactivity during crosslinking. Therefore, the effect of initiator concentration on gelation was investigated with SPELA-nL14.8 as the least favorable case and LPELA-nL7.4 with a similar lactide segment length was used for comparison. It is well established that the viability of cells encapsulated in synthetic gels is adversely affected by low molecular weight species such as initiator, crosslinker, and small-molecule monomers that cross the cell membrane. Based on previous studies, photoinitiator concentrations >2 wt % (based on the weight of macromonomer) significantly decreased viability of the seeded cells. Therefore, the effect of initiator on gelation of SPELA and LPELA was tested with concentrations <1.4 wt %. In the absence of initiator, the shear moduli of LPELA-nL7.4 and SPELA-nL14.8 systems were 0.13 and 0.08 Pa, respectively, and G″/G′) were 5.3 and 1.8. Therefore, the precursor solutions without initiator did not form a hydrogel network with UV irradiation (no gelation time in FIG. 12B). For each initiator concentration, the modulus of SPELA gel was significantly higher than that of LPELA. The modulus of the hydrogels showed a maximum at 0.38 wt % initiator concentration for both LPELA-nL7.4 and SPELA-nL14.8 macromonomers. As the initiator concentration was increased from 0.08 to 0.38 wt %, the modulus of LPELA and SPELA gels initially increased from 13.1±3.0 to 20.2±4.1 kPa and from 17.5±2.0 to 37.5±2.5 kPa, respectively. After that, the modulus decreased to 17.2±2.1 and 32.9±2.5 kPa for LPELA and SPELA hydrogels, respectively, when the initiator concentration was increased to 1.31 wt %. The modulus of the gels did not change for initiator concentrations >0.8 wt %. The initial increase in the gel modulus with initiator concentration can be attributed to an increase in propagation rate (Rₚ) given by:

$$\begin{matrix}{R}_{p} = {{K}_{p} [ AC ] [ \frac{{R}_{i}}{{K}_{i}} ]}^{1 / 2} & ( 17 ) \\ {R}_{i} = φɛ {I}_{0} δ [ I ] & ( 18 )\end{matrix}$$

where K_{P} and Kᵢ are the rate constants for chain propagation and termination respectively, Rᵢ the radical initiation rate, [AC] is the concentration of unreacted acrylates, φ is initiation efficiency, ε is molar extinction coefficient, I₀ is the intensity of incident radiation, δ is sample thickness, and [I] is photoinitiator concentration. According to the equation, the rate of radical production increased with increasing initiator concentration to 0.38 wt % leading to a higher propagation rate of acrylates and higher extent of crosslinking. For initiator concentrations exceeding 0.38 wt %, the probability of formation of more than one radical on the same macromonomer increased, which led to the formation of intra-molecular crosslinks, as opposed to inter molecular crosslinks, and cluster formation and a decrease in storage modulus. For initiator concentrations >0.8 wt %, the increase in propagation rate was offset by the increase in the rate of intra-molecular crosslinking, resulting in no change in modulus with increase in initiator concentration.

As the initiator concentration was increased from 0.08 to 0.78 wt % (see FIG. 12B), gelation time of LPELA and SPELA macromonomers decreased from 140±5 to 45±1 s and from 200±9 to 42±2 s, respectively. At low initiator concentrations (0.08 to 0.23 wt %), SPELA had higher gelation times than LPELA but the two macromonomers reached similar gelation times for concentrations >0.5 wt %. As described in the following paragraph, the total volume of the hydrophobic micelles in SPELA-nL14.8 was higher than LPELA-nL7.4. As a result, at low initiator concentrations, it was more likely for the SPELA polymerization reaction to become diffusion controlled than LPELA, leading to a higher gelation time for SPELA. As the initiator concentration was increased above 0.23 wt %, the reaction became less controlled by diffusion, leading to comparable gelation times for LPELA and SPELA at higher concentrations, In addition, the higher concentration of reactive acrylates in SPELA was offset by the higher probability of intra-molecular crosslinking, leading to comparable gelation times for SPELA and LPELA at initiator concentrations >0.23 wt %. In the sections that follow, the initiator concentration of 0.75 wt % was used, unless otherwise specified, to have low gelation times as well as high shear storage moduli.

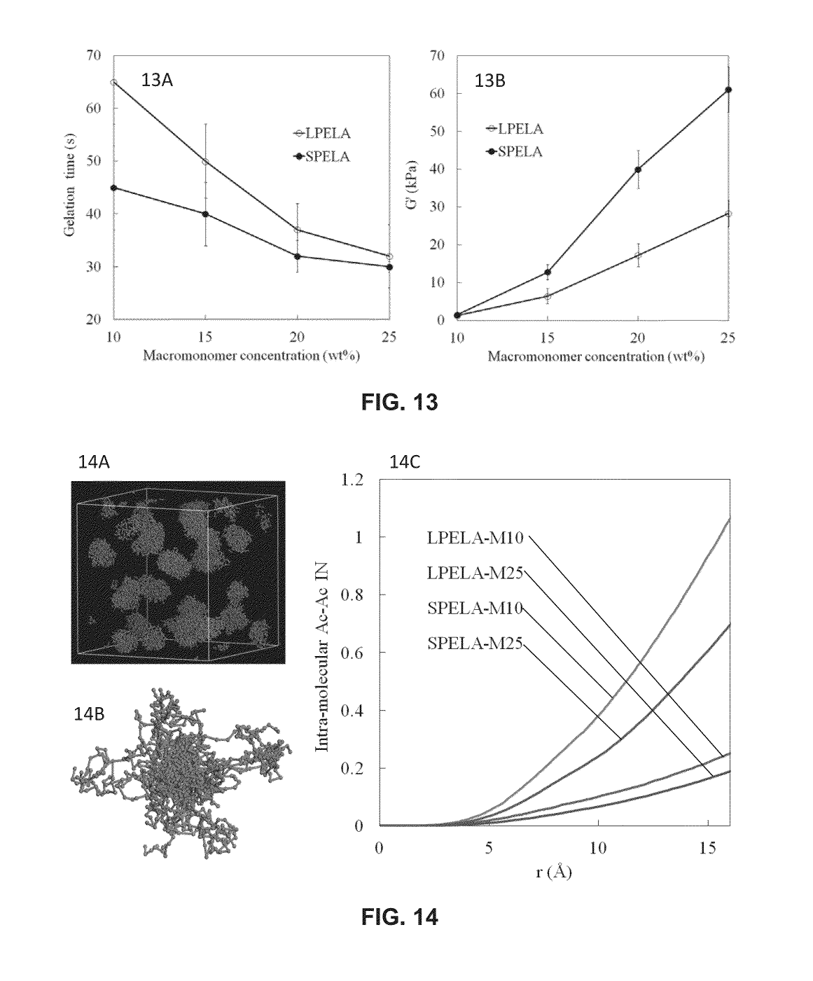

The effect of macromonomer concentration in the hydrogel precursor solution on gelation time and modulus of LPELA-nL7.4 and SPELA-nL14.8 hydrogels is shown in FIG. 13A and FIG. 13B, respectively. The gelation time of SPELA gels decreased from 45±8 to 30±1 s while LPELA gels decreased from 65±8 to 32±6 s as the macromonomer concentration increased from 10 to 25 wt %, The storage modulus of LPELA and SPELA gels increased from 1.2±0.5 to 28.3±3.5 kPa and from 1.5±0.6 to 61.0±6.2 kPa, respectively, with increasing macromonomer concentration from 10 to 25 wt %. In the absence of a crosslinker, the dependence of propagation rate of the crosslinking reaction on the concentration of reactive acrylate groups is given by the above equation. The gelation time of SPELA was slightly lower than LPELA at low acrylate concentrations (<0.04 mol/L) and the modulus of SPELA was higher than LPELA at high acrylate concentrations (>0.08 mol/L). According to the equation, higher acrylate concentrations increase propagation rate and density of crosslinks for both LPELA and SPELA macromonomers. Therefore, the decreasing trend of gelation time with increasing SPELA and LPELA macromonomer concentrations as well as the lower gelation time of SPELA at constant concentration (see FIG. 13A) can be attributed to the higher concentration of acrylates.

The shear modulus of an ideal network is proportional to the density of elastically active links according to the theory of rubber elasticity:

G=v_{E}RT (19)

where v_{E} is the concentration of elastically active chains, R is the gas constant and T is absolute temperature. The higher acrylate densities for SPELA compared to LPELA and higher macromonomer concentrations led to higher propagation rates, higher density of elastically active chains, and higher modulus. Furthermore, due to a larger average distance between the macromonomers at low concentrations, the probability of intra-molecular crosslinks that lead to loop formation and cyclization was higher. Since intra-molecular crosslinks are not elastically active and do not contribute to the network modulus, the higher density of acrylates in SPELA was offset by higher intra-molecular crosslinks, leading to a smaller difference between the moduli of SPELA and LPELA gels at low concentrations (see FIG. 13B). As macromonomer concentration was increased, the probability of intra-molecular links decreased, leading to higher fraction of elastically active crosslinks in SPELA and larger difference between the moduli of SPELA and LPELA.

As shown in FIG. 13B, the shear modulus of SPELA gels was significantly higher than those of LPELA for all macromonomer concentrations. For example, the ratio of G′_{SPELA}/G′_{LPELA} increased from 1.2 at 10 wt % macromonomer concentration to 2.2 at 25 wt %. As mentioned, the lower values of G′_{SPELA}/G′_{LPELA} at low concentrations can be attributed to the macromonomer architecture and its effect on nano-scale structure formation. DPD simulation of the macromonomers showed a uniform distribution of Ac beads over the simulation box in the absence of L beads. With the addition of lactide segments to the macromonomer, the hydrophobic L and Ac beads aggregated and formed core of the micelles, as shown in FIG. 14A for SPELA-nL14.8-M20 (W, EO and SPEGc beads are not shown for clarity). The cross-section of one of the micelles corresponding to the DPD simulation in FIG. 14A is shown in FIG. 14B. According to simulation results, hydrophilic ethylene oxide segments (EO beads) of the macromonomers surrounded the core and formed the micelle's corona. Localization of Ac beads in the micelles' core led to the formation of elastically active inter-molecular and elastically inactive intra-molecular crosslinks, To quantify the proximity of Ac beads to other beads on the same macromonomer, the average number of intra-molecular acrylate beads [(IN_{intra-Ac}(R)] in a sphere of radius R around an Ac bead or the running integration number (IN) is calculated by:

$${IN}_{intra - Ac} ( R ) = 4 {πρ}_{Ac 0} {\int }_{0}^{R} {g}_{intra - Ac} ( r ) {r}^{2} d r \qquad ( 20 )$$

where ρ_{Ac0} is the overall number density of Ac beads and g_{intra-Ac}I is the radial distribution function of intra-molecular acrylates in a shell of infinitesimal thickness at distance r from each Ac bead, located at the origin. The IN_{intra-Ac} profile for LPELA and SPELA hydrogels at 10% and 25% macromonomer concentrations is shown in FIG. 14C. The IN_{intra-Ac} for LPELA and SPELA at R=15 Å decreased from 0.22 to 0.16 and from 0.93 to 0.60, respectively, with increasing macromonomer concentration from 10% to 25%. Therefore, the probability of intra-molecular reaction for SPELA was more sensitive to macromonomer concentration than that of LPELA. This effect is reflected in the higher G′_{SPELA}/G′_{LPELA} ratios at higher macromonomer concentrations.

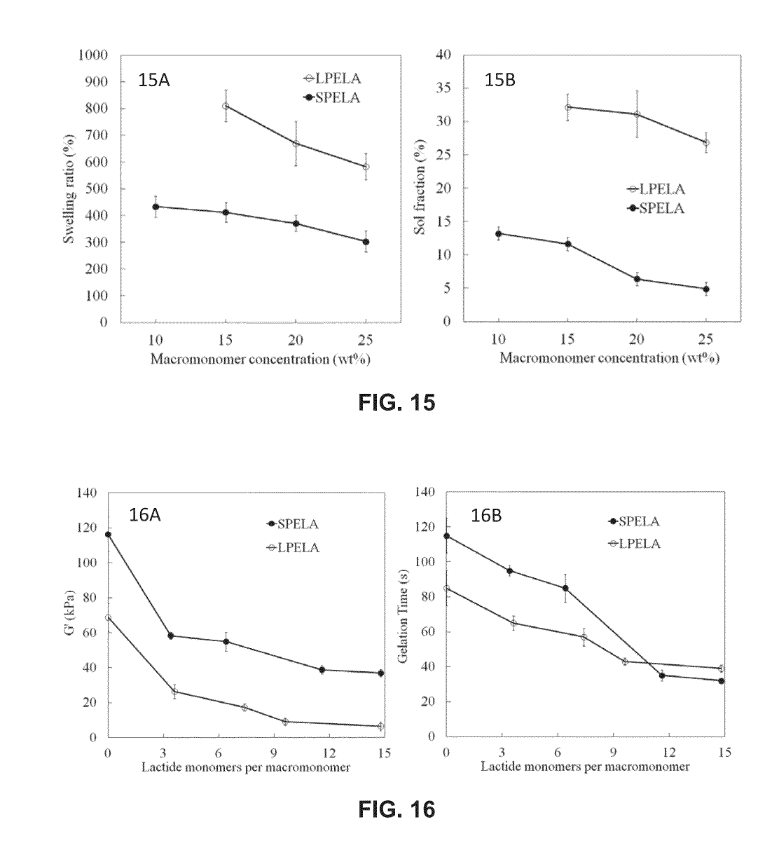

The effect of macromonomer concentration on swelling ratio and sol fraction of LPELA-nL7.4 and SPELA-nL14.8 hydrogels is shown in FIG. 15A and FIG. 15B, respectively. The LPELA-nL7.4-M10 samples disintegrated upon removal from pettier plate of the rheometer, so the swelling and sol fraction of that sample was not measured. The swelling ratio of SPELA and LPELA gels decreased from 430 to 300% and from 810 to 580%, respectively, as the macromonomer concentration increased from 10/15% to 25%. The higher swelling ratio of LPELA gels compared to SPELA was related to the lower fraction of hydrophobic segments per macromonomer in LPELA along with lower crosslink density of LPELA gels. Sol fraction of LPELA gels decreased from 32.1±2.0% to 26.8±1.5% as concentration increased from 15 to 25%. The sol fraction of SPELA decreased from 13.2±1.1 to 11.6±1.1, 6.4±1.0 and 4.9±0.9 as macromonomer concentration increased from 10 to 15, 20 and 25%, respectively. Sol fraction of the hydrogels decreased by 21, 4.8 and 5.4 folds by changing macromonomer architecture from linear to star at 15, 20 and 25% concentration, respectively. The significantly lower sol fraction of SPELA hydrogel compared to LPELA was due to the higher concentration of reactive acrylate groups in SPELA at the same macromonomer concentration. The decreasing trend in sol fraction with macromonomer concentration was related to the decrease in intermolecular distance between the acrylate groups with concentration, which enhanced the probability of formation of elastically active crosslinks.

FIG. 16A and FIG. 16B show the effect of number of lactide monomers per macromonomer on shear modulus and gelation time, respectively, for LPELA-M20 and SPELA-M20 hydrogels. G′ of the hydrogels initially decreased from 116±10 kPa to 58±2 kPa and from 69±8 kPa to 26±4 kPa with the addition of 3.6 and 3.4 lactide monomers per macromonomer (nL) for SPELA and LPELA hydrogels, respectively, corresponding to 1.8 and 0.85 monomers per arm of LPELA and SPELA hydrogels. Afterward, G′ decreased at a slower rate to 6.5±2.5 kPa and 37.0±2.0 kPa for LPELA and SPELA gels, respectively, when nL increased to 14.8. The gelation time of SPELA hydrogel decreased from 115±10 s to 32±1 s while that of LPELA decreased from 85±10 s to 39±2 s with increasing nL from 0 to 14.8. The decrease in G′ and gelation time of the hydrogels with increasing lactide segment length is related to aggregation and micelle formation of the macromonomers in aqueous solution, The size of the micelles' core increased with increasing lactide segment length. At any given time, macromonomer arms can have one of three conformations, namely bridge, which connects two different micelle cores, loop with at least two arms of a macromonomer in the same micelle, and free arm which is in solution (not part of the micelles). The dynamic nature of these conformations leads to the formation of a transient physical network in the precursor solution. In SPELA-nL0 system, the gelation time was high due to a relatively large average distance between the uniformly distributed acrylate groups. The localization of reactive acrylate groups in the micelles core with increasing lactide content decreased the average distance between the acrylate groups. As a result, the reaction rate between acrylates increased with increasing lactide segment length. In addition, the lifetime of the bridging arms in the core increased with increasing lactide segment length. Those factors worked together to decrease gelation time with increasing lactide content of the macromonomers. At the same time, due to the very low ater content of the micelles, mobility of the acrylate groups and diffusion of initiator in the micelles significantly decreased with increasing micelles' core size. As a result, a fraction of the acrylates, trapped in the micelles' core, did not react to form elastically active crosslinks, which led to a decrease in hydrogel modulus with increasing macromonomer lactide content.

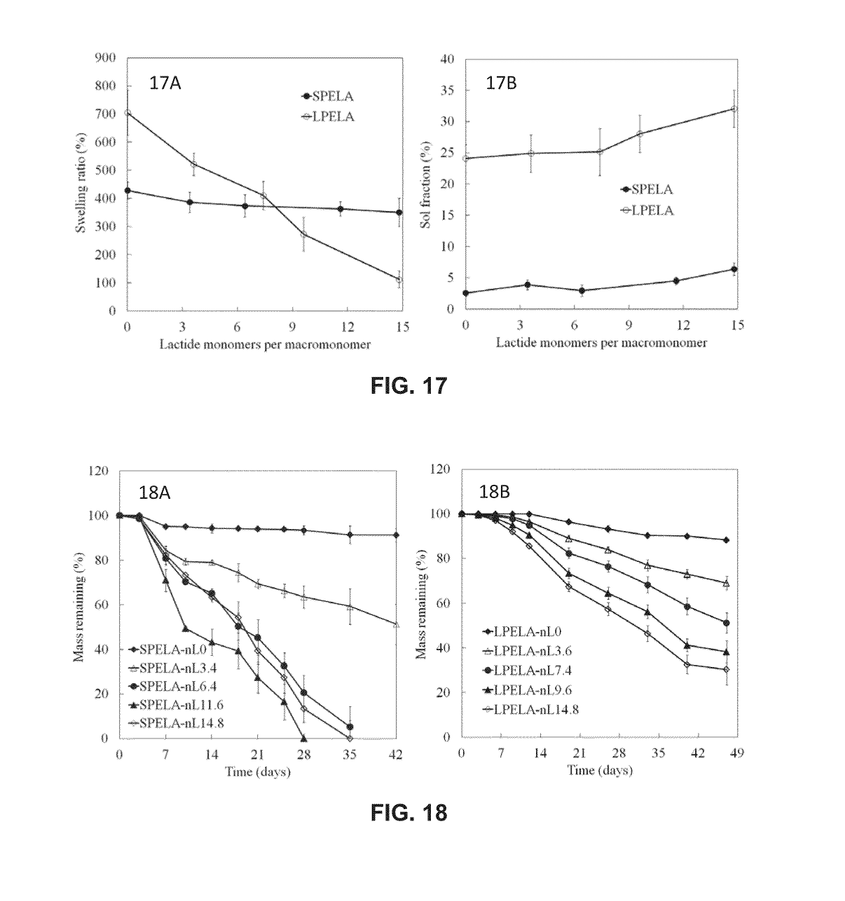

The effect of number of lactides per macromonomer on sol fraction and swelling ratio of LPELA and SPELA hydrogels is shown in FIG. 17A and FIG. 17B, respectively. The swelling ratio of LPELA gels decreased dramatically from 700% to 110% when nL increased from 0 to 14.8 while that of SPELA decreased slightly from 430% to 350%. The decrease in network density with increasing nL had a positive effect on swelling ratio while the increase in hydrophobicity with increasing nL had a negative effect on swelling ratio. The molecular weight between crosslinks, M̅_̅{̅c̅}̅, for an ideal network in the affine model is given by:

$$\frac{1}{\overline{{M}_{c}}} = ( \frac{F}{F - 2} ) \frac{1}{\overline{{M}_{n}}} + \frac{G \overline{v}}{RT} {( \frac{{v}_{2 , r}}{{v}_{2 , s}} )}^{1 / 3} \qquad ( 21 )$$

where F is functionality of crosslinks (3 for crosslinks at chain ends), M̅ₙ̅ is macromonomer molecular weight, G is network modulus, v̅ is specific volume of the macromonomer, and v_{2,r} and v_{2,s} are the macromonomer volume fraction in the crosslinked gel before and after swelling equilibrium, respectively. According to the above equation, in the absence of inhomogeneity in the gel (e.g. PEG networks with no lactide segments), M̅_̅{̅c̅}̅ increases and G decreases with M̅ₙ̅. Therefore, the higher swelling of LPELA-nL0 compared to SPELA-nL0 is attributed to the lower crosslink density of the linear LPELA compared to star SPELA. In the presence of lactide, aggregation of hydrophobic segments produced micellar inhomogeneity in the network and increased hydrophobicity. Based on simulation results, water content of the hydrophobic domains was <1%. So the SPEL and LPELA gels may be better described as nanophase separated networks. The interfacial free energy of the micelles with the aqueous phase increased with increasing nL for both SPELA and LPELA macromonomers. However, the micelles in SPELA had a lower interfacial energy than LPELA. The ethylene oxide chains in star SPELA macromonomer provided greater surface coverage for micelles' core, as predicted by DPD simulation (data not shown), thus lowering the interfacial energy of SPELA compared to LPELA. In addition, the lower radius of gyration of star SPELA led to higher steric repulsion between the EO units in the macromonomer, which reduced the equilibrium core size and the average distance between the micelles in SPELA compared to LPELA. Therefore, as nL increased. SPELA macromonomers formed smaller micelles closer to each other while LPELA formed larger micelles farther away from each other. The higher inter-micellar distance in LPELA and extension of the bridging arms of the cores sharply reduced swelling ratio of LPELA as nL was increased (see FIG. 17A) while the swelling ratio of SPELA was unaffected. SPELA hydrogel had a significantly lower sol fraction than LPELA, as shown in FIG. 8b. For example, sol fraction of LPELA and SPELA hydrogels increased from 24±2% to 32±3% and from 2.5±0.5% to 6.4±1.0% as nL increased from 0 to 14.8. This was attributed to the higher density of reactive acrylate groups in SPELA compared to LPELA, which increased the probability of incorporating macromonomers in the gel network.

The effect of lactide content per macromonomer on mass loss of the SPELA and LPELA hydrogels is shown in FIG. 18A and FIG. 18B, respectively. There was no significant difference between the mass loss curves of SPELA-nL6.4 and SPELA-nL14.8 (p=0.34) but there was a significant difference between the mass loss of all other SPELA pairs (p<0.05). There was a significant difference between the mass loss of all LPELA pairs (p<0.05). Mass loss of SPELA-nL0 and LPELA-nL0 was <10% after 42 days. For a given time, mass loss of SPELA increased with increasing nL up to nL=11.6 followed by a decrease in mass loss for higher nL values but it was higher than LPELA at any incubation time. Mass loss of LPELA increased with nL. For example, mass loss of SPELA hydrogels after 28 days changed from 7% to 37%, 80%, 100%, and 87% as the number of lactide monomers in SPELA increased from zero to 3.4, 6.4, 11.6, and 14.8, respectively, while those of LPELA increased from 7%, to 15%, 26%, 38%, and 46% as the number of lactide monomers in LPELA increased from zero to 3.6, 7.4, 9.6 and 14.8. The SPELA hydrogel with 11.6 lactides per macromonomer completely degraded after 28 days. Assuming the formation of carboxylic acid groups by degradation of lactides does not significantly affect mass loss, degradation rate of SPELA is given by:

R_{deg,SPELA}=k[—COO—][H₂O] (22)

where k is the degradation rate constant, and [—COO—] and [H₂O] are the concentrations of ester groups and water in the hydrogel, respectively. Degradation of PLA matrices is controlled by the low concentration of water in the matrix while degradation of SPELA hydrogel is controlled by relatively low concentration of degradable ester units. The decline in the concentration of ester groups in the hydrogel with degradation was offset by the increase in water content (increased swelling ratio), leading to nearly constant degradation rate, as shown in FIG. 18. The increase in mass loss with nL (up to 11.6) was attributed to the higher concentration of ester groups in SPELA hydrogel. The decrease in mass loss of SPELA for nL>11.6 was attributed to micelle formation with significantly reduced local concentration of water, leading to reduced rate of degradation.

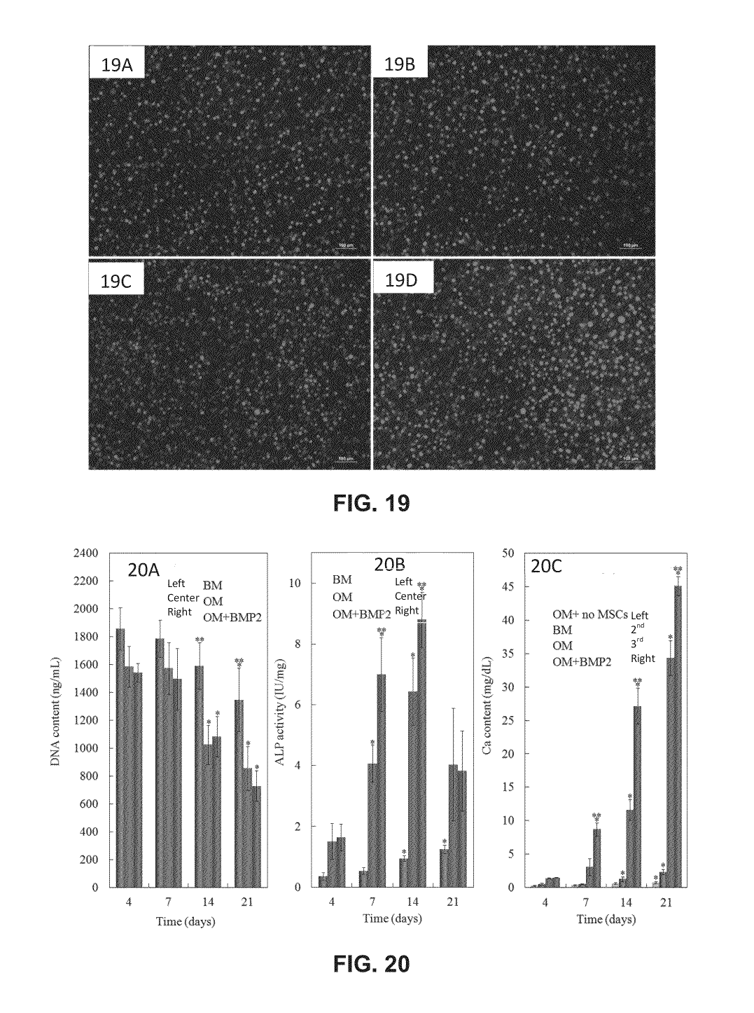

FIG. 19A through FIG. 19D show live and dead cells 1 h after encapsulation in SPELA-nL3.4, SPELA-nL6.4, SPELA-nL11.6, SPELA-nL14.8 hydrogels, respectively. Based on the images, the number of lactides per macromonomer did not have a significant effect on cell viability. Cell viability was quantified by dividing the image into smaller squares and counting the number of live and dead cells. The fraction of viable cells for SPELA-nL0, SPELA-nL3.4, SPELA-nL6.4, SPELA-nL11.6, and SPELA-nL14.8 gels was 92±3, 90±4, 92±4, 94±4, and 91±3, respectively.

SPELA-nL14.8 has the longest hydrophobic lactide segment length and highest local density of degradable ester groups, compared to other SPELA macromonomers, leading potentially to highest local changes in pH with incubation. In turn, local pH changes can lead to reduced cell viability (worst case). Therefore, SPELA-nL14.8 was selected for encapsulation and osteogenic differentiation of MSCs.

DNA content, ALPase activity, and extent of mineralization of MSCs encapsulated in SPELA are shown in FIG. 20A, FIG. 20B, and FIG. 20C, respectively. The cell free gels did not have DNA count, ALPase activity and mRNA expression but showed slight calcium content. For all time points, MSCs in BM had higher cell count than those cultured in OM, and the addition of BMP2 did not affect DNA count. At day 21, DNA count of MSCs in BM decreased slightly (statistically significant) compared to days 4-14. For days 14 and 21, DNA content of MSCs in OM and OM+BMP2 groups was statistically lower than days 4 and 7 (indicated by a star in FIG. 20A), and statistically lower than BM group. This trend is consistent with previous reports that cell number decreases with differentiation of MSCs in osteogenic medium.

MSCs in BM group (FIG. 19 and FIG. 20) had significantly lower ALPase activity, calcium content, and expression of osteogenic marker than OM and OM+BMP2. ALPase activity of both groups (with and without BMP2) significantly increased (indicated by one star) from day 4 to 7 and 14 and returned to the baseline level after 21 days. This was consistent with our previous results that the peak ALPase activity is the start of mineralization. ALPase activity of BMP2 group at days 7 and 14 was significantly higher than that of OM (without BMP2, indicated by two stars). Calcium content of both groups increased significantly on days 7-21, compared to day 4 (indicated by one star). However, calcium content of the BMP2 group was significantly higher that the OM group for days 7-21 (indicated by two stars). For example, calcium contents of the BMP2 group after 7, 14, and 21 days were 8.7±1.0, 27.1±2.7, and 45.1±1.4 mg/dL, respectively, while those of OM were 3.1±1.3, 11.6±1.6, and 34.4±2.6 mg/dL. Results in FIG. 20C demonstrates that the calcium content of MSCs in OM and OM+BMP2 groups was due to osteogenic differentiation and mineralization of MSCs, and not due to the calcium in osteogenic media.

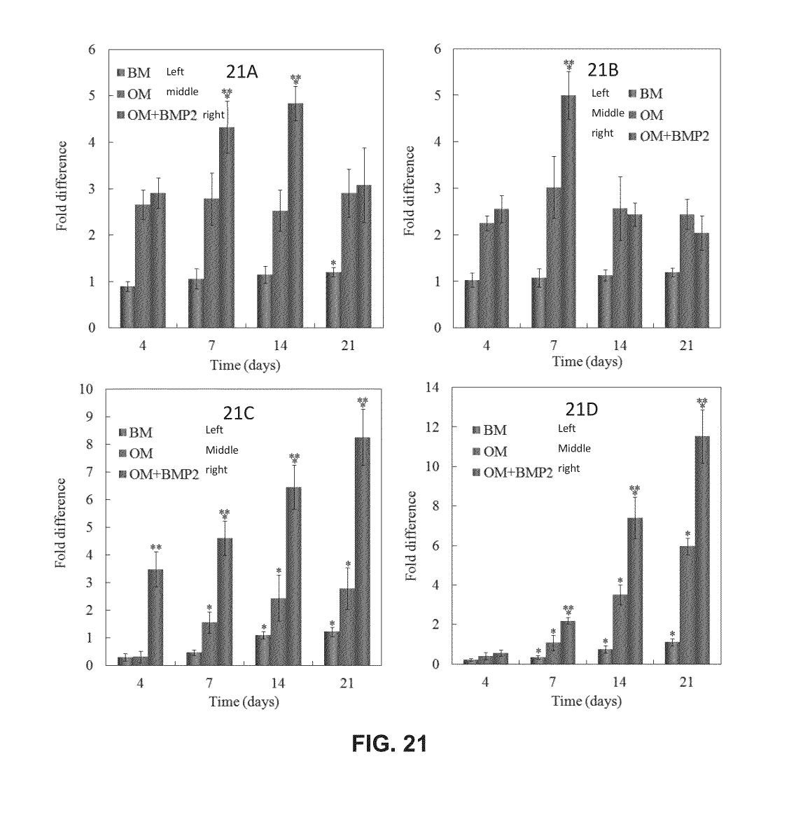

mRNA expression levels of DIx5, Runx2, OP, and OC of the MSCs for both groups (with and without BMP2) are shown in FIG. 21A through FIG. 21D with incubation time. The fold differences in mRNA expression of the markers are normalized to those at time zero. BMP2 protein forms complexes with type I and type II BMP2 receptors (BRI and BRII) on the surface of MSCs, which activates the Smad-dependent and Smad-independent p38 pathways as well as internalization of the receptors. The expression of DIx5 and Runx2 is the early event in the BMP2 signaling cascade. DIx5 regulates the activity of osteogenic master transcription factor Runx2 by Smad-dependent pathways, which in turn drives the expression of osteogenic genes. The expressions of DIx5 and Runx2 were up-regulated for all time points (see FIG. 21A and FIG. 21B) for both OM and BMP2 groups. However, for BMP2 group, there was a sharp increase in the expression of DIx5 on days 7 and 14 and the expression of Runx2 on day 7. mRNA expression of OP and OC increased gradually for both OM and BMP2 groups with incubation time, but the fold difference was significantly higher for BMP2 group at each time point. For example, the fold differences in OC mRNA expression for OM group increased from 0.4±0.2 to 1.1±0.4, 3.5±0.5, and 6.0±0.4 after 4, 7, 14, and 21 days of incubation, respectively, while those for BMP2 group increased from 0.6±0.2, 2.2±0.2, 7.4±1.1, and 11.5±1.3. Results in FIG. 20 and FIG. 21 taken together demonstrate that the encapsulated MSCs in OM and OM+BMP2 groups differentiated in osteogenic media while those incubated in BM did not undergo osteogenic differentiation. Yang et al. investigated osteogenic differentiation of MSCs encapsulated in RGD-conjugated PEG hydrogels. The calcium ntent of MSCs with optimum RGD density after 21 days was 0.00008 mg/mg DNA, compared to 0.057 mg/mg DNA in this work. The higher calcium content in this work may be related to the degradable nature of SPELA matrix, leading to increase in water content, free volume, and greater cell-matrix interaction with incubation time. The addition of inductive factors like BMP2 significantly enhanced differentiation and mineralization of MSCs.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A biocompatible hydrogel network comprising:
a crosslinked macromonomer, the macromonomer including a biocompatible polymer and a hydrophobic segment at the termini of the biocompatible polymer, the hydrophobic segment including no more than 5 hydrophobic monomers; wherein
the hydrogel network comprises a micelle that includes a core and comprises the crosslinked macromonomer such that the hydrophobic segment is sequestered in the core of the micelle.

2. The biocompatible hydrogel network of claim 1, wherein the hydrophobic monomers are hydroxy acid monomers.

3. The biocompatible hydrogel network of claim 1, wherein the hydroxy acid monomers comprise glycolide, lactide, dioxanone, ε-caprolactone, hydroxy butyrate, valcrolactone, malonic acid, or mixtures thereof.

4. The biocompatible hydrogel network of claim 1, wherein the hydrophobic monomers comprise lipid monomers, anhydride monomers, orthoester monomers phosphazene monomers, hydroxy acid monomers, or mixtures thereof.

5. The biocompatible hydrogel network of claim 1, wherein the crosslinked macromonomer is crosslinked via acrylate functionality.

6. The biocompatible hydrogel network of claim 1, the crosslinked network further comprising a crosslink initiator, wherein the crosslink initiator is sequestered within the core of the micelle.

7. The biocompatible hydrogel network of claim 1, wherein the biocompatible polymer is polyethylene glycol, polyvinyl alcohol, polyhydroxyethyl methacrylate, polyvinylpyrrolidone, polyacrylic acid, polymethacrylate, polyacrylamide, or a polymethyl methacrylate.

8. The biocompatible hydrogel network of claim 1, wherein the biocompatible polymer is a linear, branched, or star polymer.

9. The biocompatible hydrogel network of claim 1, wherein the hydrophobic segment includes from 1 to 3 hydrophobic monomers.

10. The biocompatible hydrogel network of claim 1, wherein the network exhibits a linear degradation rate over time.

11. The biocompatible hydrogel network of claim 1, further comprising a biologically active material.

12. The biocompatible hydrogel network of claim 11, wherein the biologically active material comprises a cell, a tissue explant, or a cellular extract.

13. The biocompatible hydrogel network of claim 12, further comprising one or more signal molecules.

14. The biocompatible hydrogel network of claim 1, wherein the hydrogel network has a compressive modulus of from about 50 kilopascals to about 1000 kilopascals.

15. The biocompatible hydrogel network of claim 1, wherein the hydrogel network has a swelling ratio of from about 250% to about 850%.

16. The biocompatible hydrogel network of claim 1, wherein the hydrogel network has a sol fraction of from about 2% to about 10%.

17. The biocompatible hydrogel network of claim 1, wherein the micelle has a cross sectional dimension of from about 1 nanometer to about 5 nanometers.

18. A method for forming a biocompatible hydrogel network comprising:
extending a chain of a biocompatible polymer with a hydrophobic segment to form a macromonomer, the hydrophobic segment comprising no more than 5 hydrophobic monomers;
crosslinking the macromonomer to form the hydrogel network, the crosslinked macromonomer forming a micelle that includes a core, the hydrophobic segment being sequestered in the core.

19. The method of claim 18, further comprising acrylating the macromonomer.

20. The method of claim 18, wherein the macromonomer is crosslinked by use of electromagnetic radiation.

21. The method of claim 20, wherein the electromagnetic radiation is ultraviolet radiation.

22. The method of claim 18, further comprising loading one or more biologically active materials on the hydrogel network.

23. The method of claim 22, wherein the biologically active materials comprise a cell, a tissue explant, or a cellular extract.

24. The method of claim 18, wherein the biocompatible polymer is polyethylene glycol, polyvinyl alcohol, polyhydroxyethyl methacrylate, polyvinylpyrrolidone, polyacrylic acid, polymethacrylate, polyacrylamide, or a polymethyl methacrylate.

25. The method of claim 18, wherein the biocompatible polymer is a linear, branched, or star polymer.

26. The method of claim 18, wherein the hydrophobic monomers comprise hydroxy acid monomers.

27. The method of claim 26, wherein the hydroxy acid monomers comprise glycolide, lactide, dioxanone, ε-caprolactone, hydroxyl butyrate, valcrolactone, malonic acid, or mixtures thereof.

28. The method of claim 18, wherein the hydrophobic monomers comprise lipid monomers, anhydride monomers, orthoester monomers phosphazene monomers, hydroxy acid monomers, or mixtures thereof.

29. The method of claim 18, wherein the macromonomer crosslinks in a period of time from about 20 seconds to about 180 seconds.

30. The method of claim 18, wherein the macromonomer crosslinks in a period of time that decreases with increase in the number of hydrophobic monomers in the hydrophobic segment.
